(19) United States
(12) Patent Application Publication
(10) Pub. No.: US 2002/0045190 A1
(43) Pub. Date: Apr. 18, 2002

(54) Encoding methods using up-converting phosphors for high-throughput screening of catalysts

(76) Inventors: Robert B. Wilson JR., Palo Alto, CA (US); Christopher D. Tagge, San Carlos, CA (US); Naheed Mufti, Castro Valley, CA (US)

Correspondence Address: MORGAN, LEWIS & BOCKIUS, 1800 M STREET NW, WASHINGTON, DC, 20036-5869, US

(21) Appl. No.: 09/901,912

(22) Filed: Jul. 11, 2001

Related U.S. Application Data
(60) Provisional application No. 60/217,404, filed on Jul. 11, 2000.

(51) Int. Cl. G01N 33/53; G01N 33/543; C08F 2/00

(52) U.S. Cl. 435/7.1; 436/523; 526/59

(57) ABSTRACT

This invention relates to encoding methods using up-converting phosphors for high-throughput screening of catalysts. In particular, the invention relates to polymerization catalysts. In one embodiment of the invention, a monomer is combined with at least one labeled catalyst particle. The labeled catalyst particle comprises an up-converting phosphor label and a catalyst. The up-converting phosphor label identifies a particular catalyst. The monomer is polymerized to form a polymer bead surrounding the labeled catalyst particle. According to the invention, it is possible to combine two or more labeled catalyst particles in a commercially relevant large scale reactor to obtain direct comparison of different catalysts, and to avoid reactor effects that may occur when performed on the microscale. After the reaction, the resulting polymer beads are sorted, based on their size and/or phosphor labels. The polymer beads may also be further characterized to determine various physical and chemical properties of the resultant polymer.

This application claims priority under 35 U.S.C. § 119(e) to U.S. Provisional Application No. 60/217,404, filed Jul. 11, 2000, herein incorporated by reference in its entirety.

### FIELD OF INVENTION

The invention relates to encoding methods using up-converting phosphors as labels for catalysts and methods for screening catalysts. The encoding methods may be applied to combinatorial and/or high-throughput techniques, and used to study catalysis in a variety of reactions.

### BACKGROUND OF THE INVENTION

Due to the complexity of most catalytic reactions, as well as the lack of empirical rules to predict catalytic activity and the properties of the resulting product, catalyst research and development is often a slow process. Combinatorial chemistry and high-throughput methods have been developed to synthesize and/or test large numbers of compounds. However, in typical combinatorial or high-throughput techniques, reactions are often run at the microscale, and often must be run in apparatus designed specifically for combinatorial synthesis. This may be a problem for certain types of reactions, such as complex polymerization reactions, where the catalyst performance is often profoundly affected by reactor size, shape and reaction conditions. Variations in catalyst performance are commonly observed between different reactors, even on large scale. Inevitably, variations in reaction conditions also occur over time, even when doing sequential reactions using the same reactor, which can prevent an accurate comparison between catalysts. Therefore, what is needed in the art is a method for accelerating catalyst development by conducting up to several hundreds of polymerization reactions simultaneously and a method to directly screen the activities of several catalysts under identical reaction conditions in a single, commercially relevant reactor. The methods should also be applicable to large scale reactions. This invention answers that need.

When screening large numbers of compounds in a collection of compounds, such as combinatorial libraries, various methods have been developed to identify individual members of the collection or library. These methods include the development of “tags” or “labels”. For a general discussion of tagging and encoding strategies, see Wilson, S. R. and Czamik, A. W., Eds., Combinatorial Chemistry, John Wiley & Sons, New York, 1997, which is hereby incorporated by reference in its entirety. Other encoding strategies are described, for example, in U.S. Pat. Nos. 5,789,172; 5,786,219; and 5,565,324; and WO 99/24822 which are hereby incorporated by reference in their entirety. A method for labeling polystyrene-supported catalysts using mass spectroscopic detection of amine tags is described in T. R. Boussie et al. Angew. Chem. Int. Ed., 37:3272-3275, (1998) incorporated by reference. However, many of these encoding strategies using “tags” or “labels” often require additional synthetic steps to attach the tags, or cleavage steps to read the tags and identify the product, which reduces efficiency and increases costs. Some of the encoding methods may also limit the types of synthetic reactions which may be used, due to incompatibility of the “tags” or “labels” with a broad range of reagents, temperatures, pressures, and other reaction conditions. What is needed in the art is an encoding method, where the label does not require numerous additional synthetic or cleavage steps, and where the label is compatible with a broad range of reaction conditions. This invention answers this need.

While not applied to combinatorial or high-throughput screening, up-converting phosphors are generally described in U.S. Pat. Nos. 5,891,656; 5,736,410; 5,698,397; and 5,674,698, which are hereby incorporated by reference in their entirety. These patents describe up-converting phosphors and their use as labels in clinical and biological applications, such as assay methods for detecting soluble, suspended, or particulate substances or analytes such as proteins, carbohydrates, nucleic acids, bacteria, viruses, and eukaryotic cells and compositions and methods that include luminescent (phosphorescent or fluorescent) labels. For instance, an analyte (e.g., polypeptides, polynucleotides, etc.) is detected by adding a labeled probe (e.g., an antigenic epitope, etc.) which binds the analyte. In addition, up-converting phosphors are also known for their applications in various devices, such as fluorescent lamps, plasma-panel display gas-discharge cells, electron-beam display devices, and other emissive displays, such as those described in U.S. Pat. No. 6,013,199 which is hereby incorporated by reference in its entirety. However, as mentioned above, phosphor labels have not been applied to combinatorial or high-throughput screening of catalysts. There exists a significant need in the art for labels and detection methods for catalytic reactions, which permit sensitive optical and/or spectroscopic detection of specific label signal(s) with essentially total rejection of nonspecific background noise, and which are compatible with a variety of reaction conditions. This invention answers this need.

The articles and patents discussed above are provided solely for their disclosure prior to the filing date of this application for patent. Such discussion is not an admission that the inventors of this application are not entitled to antedate any such disclosure by virtue of a prior invention or otherwise.

### SUMMARY

This invention relates to various methods for encoding catalysts, using various combinations of spectrally distinct up-converting phosphor labels. The phosphors are incorporated into individual catalyst or support particles (e.g., using precipitation techniques) to form labeled catalyst particles, and then the labeled catalyst particles are used in a variety of chemical reactions. A parameter or property of the reaction is then monitored or measured. The up-converting phosphor label is used to match a particular catalyst with the measured parameter or property allowing the activity of the various labelled catalysts to be studied or determined.

For example in a polymerization reaction, the resulting polymer product grows around the labeled catalyst, thereby retaining the phosphor label. The corresponding catalyst is therefore quickly and easily identifiable. The size or weight of the polymer particles may be measured by methods known in the art to determine catalyst activity.

In one embodiment of this invention, polymerization catalysts may be labeled with up-converting phosphors and used in a polymerization reaction for rapid screening of catalyst activity. This embodiment comprises combining a monomer (or co-monomers) with at least one labeled catalyst particle. The labeled catalyst particle comprises a catalyst and one or more up-converting phosphor labels, which identify the catalyst, and optionally, a support material. The up-converting phosphor label comprises at least one rare earth ion, and a phosphor host material. The monomer is polymerized, to form a polymer bead around the labeled catalyst particles. The resulting polymer beads may then be sorted by size, bulk density, or the phosphor label that identifies the catalyst. In either instance, the catalyst may be identified by illuminating a polymer bead with illumination radiation, and detecting the emission radiation of at least one emission wavelength, which is characteristic of the phosphor label associated with the catalyst. The activity of the catalysts may be determined or compared, for example, by measuring the size or weight of the polymer particle or by measuring some other property of the polymer itself. Thus a large number of catalysts may be screened and/or characterized in an efficient manner.

Another embodiment of this invention relates to a method of screening or optimizing the solid support material for a supported catalyst. For instance, the polymerization reaction is carried out using a labeled catalyst particle, where the labeled catalyst particle contains an up-converting phosphor label that identifies a particular support material. The up-converting phosphor label comprises at least one rare earth ion and at least one phosphor host material. Next, a monomer or mixture of co-monomers is polymerized to form a polymer bead around the labeled catalyst particle. The resulting polymer beads may then be sorted, e.g., based on size or based upon the phosphor label. The up-converting phosphor label may be identified by illuminating the resultant polymer bead with illumination radiation and detecting the emission radiation of at least one emission wavelength, which is characteristic of the phosphor label associated with each solid support material. The activity of the catalyst on the particular support may then be determined by the methods discussed above.

This invention also relates to compositions comprising at least one up-converting phosphor, a catalyst, and optionally a catalyst support material. In the composition, the up-converting phosphor may be used to label (encode) the catalyst, the support material, or both. The up-converting phosphor comprises at least one rare earth ion, and at least one phosphor host material. The catalyst and support material may be any catalyst or support material. This embodiment of the invention also relates to a composition that comprises a polymer bead surrounding a labeled catalyst particle.

When using traditional combinatorial methods, compounds are often synthesized on the microscale or in apparatus specifically designed for combinatorial chemistry, which may affect the activity and other properties of the catalyst. However, an advantage of the invention is that the reactions may be run on any scale, ranging from microscale to large scale, e.g. a pilot plant scale. Also, according to an embodiment of the invention, the labeled catalysts may be mixed or combined and directly compared in commercially relevant and readily available reactors, as opposed to the more expensive microscale reactors. Catalyst performance is often profoundly affected by reactor size, shape and reaction conditions. Variations in catalyst behavior are observed in different reactors, even on large scale, which prevents an accurate comparison of different catalysts. Variations in reaction conditions also occur over time, even when using the same reactor in a sequential process, which also prevents an accurate comparison between catalysts. Therefore, this invention may eliminate reactor effects and reduce capital costs, as well provide a direct comparison side-by-side comparison of different catalysts under identical reaction conditions.

The up-converting phosphor bead based polymerization catalyst screen is used to rapidly screen supported and unsupported polymerization catalysts that form polymer beads around the labeled catalyst particle. For ease of analysis, the polymer beads should generally be non-aggregating and permit easy separation and sorting. The up-converting phosphor label identifies the catalyst particle embedded within the polymer bead. The catalyst activities are then judged from the size and/or weight of the resulting polymer or by measuring polymer properties known in the art, such as glass transition temperature, melt flow rate, molecular weight, molecular weight distribution, stereoregularity, crystallinity. The polymer beads may also be sorted and/or further analyzed, by combinatorial or traditional techniques, to determine other chemical and physical properties of interest.

Advantages of the invention include the ability to identify polymer beads without having to cleave the phosphor label from the product, i.e., the polymer beads that are produced. Additionally, up-converting phosphors can be incorporated into a wide range of materials, including traditional commercial catalyst supports such as silica and magnesium chloride. Generally, up-converting phosphors are chemically and thermally inert, and will not interfere with the behavior of polymerization catalysts. As described above, other advantages of the invention include, but are not limited to: screening multiple catalysts simultaneously for direct comparison of catalyst activity, eliminating reactor effects observed for small-scale polymerization or sequential reactions, analyzing catalysts in commercially relevant, readily available reactors, and sorting the resulting polymer beads for further analysis of polymer properties.

Any of the embodiments of this invention may be used either alone or taken in various combinations. Additional objects and advantages of the invention are discussed in the detailed description that follows, and will either be obvious from that description or may be learned by practice of the invention. Both this summary and the following detailed description are exemplary and explanatory only and are not intended to restrict the invention.

### BRIEF DESCRIPTION OF THE DRAWINGS

FIG. 1 shows a schematic representation of the up-converting phosphor bead based polymerization catalyst screen.

FIG. 2 shows a typical bead sorting system using a modified commercial cytometer.

FIGS. 3A, 3B, 3C show schematically energy state transitions in multi-photon excitation schemes.

FIG. 4A and 4B show a fluorescence-based flow cytometer modified for use with up-converting phosphors.

FIG. 5 is a schematic diagram of a flow cytometer in accordance with an embodiment of the present invention.

FIGS. 6 and 7 are plots showing the performance of phosphors in accordance with the present invention.

FIG. 8 is a schematic diagram of an embodiment of a flow cytometer.

FIGS. 9 and 10 are perspective views of the flow cytometer of FIG. 8.

FIG. 11 is a schematic diagram illustrating the forward scatter monitor of the flow cytometer of FIG. 8.

FIG. 12 is a schematic diagram illustrating a configuration of a flow cytometer using a linear array detector and dispersion element.

FIG. 13 shows a unique concentric ringed fiber optic assembly that may be used with a forward scatter & laser monitor.

FIG. 14 shows a block diagram of one embodiment of apparatus for carrying out the present invention on a sample using a microscope.

FIG. 15 is a block diagram of a reader for use with the present invention.

### DETAILED DESCRIPTION OF THE PREFERRED EMBODIMENTS

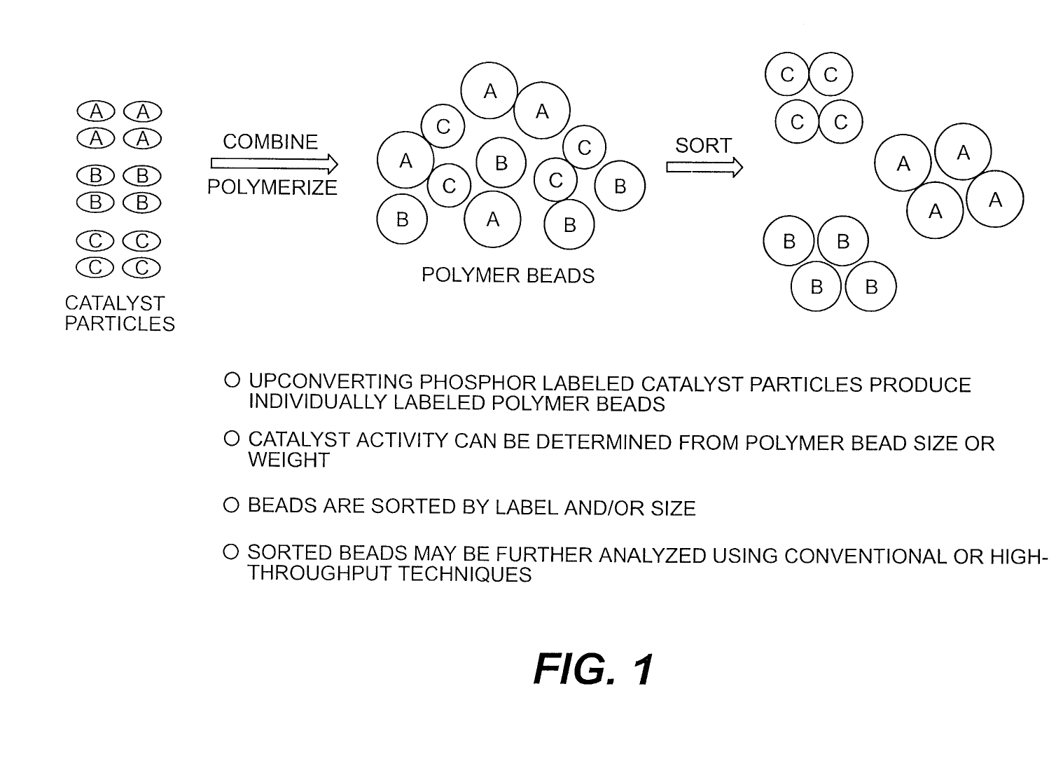

This invention relates generally to encoding methods using up-converting phosphors for high-throughput or combinatorial screening of catalysts. The phosphors are attached to the catalysts, or alternatively incorporated into solid-supported catalysts. A monomer (or mixture of co-monomers) is combined with at least one labeled catalyst particle, and optionally a co-catalyst, and/or activator. The labeled catalyst particle comprises an up-converting phosphor label and a catalyst, where the up-converting phosphor label identifies a particular catalyst. Next, the monomer is polymerized, under appropriate reaction conditions, to form a polymer bead surrounding the labeled catalyst particle. It is possible to combine two or more labeled catalyst particles in one reactor, in order to directly compare and screen different catalysts. FIG. 1 shows a schematic representation of the up-converting phosphor bead based polymerization catalyst screen.

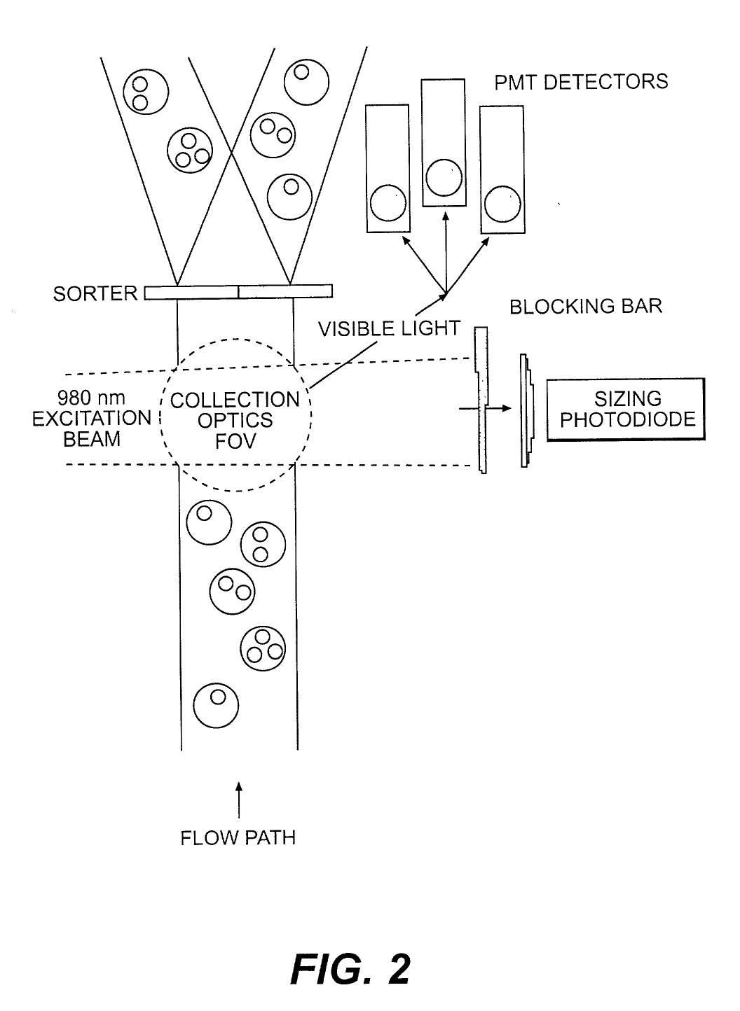

After the polymerization step, the resulting polymer beads may be sorted and/or further characterized. FIG. 2 shows a typical bead sorting system using a modified commercial cytometer.

As used herein, “label” refers to a chemical substituent that produces, under appropriate excitation conditions, a detectable optical signal. The up-converting phosphor fluorescent labels used in the invention are excited by an excitation wavelength and subsequently emit electromagnetic radiation at up-shifted frequencies i.e., at higher frequencies than the excitation radiation. The optical signal produced by an excited label is typically electromagnetic radiation in the near-infrared, visible, or ultraviolet portions of the spectrum. The labels of the invention are up-converting phosphor labels, which means that the chemical substituent absorbs at least two photons at an excitation frequency and subsequently emits electromagnetic energy at an emission frequency higher than the excitation frequency. Thus, there is generally a significant anti-Stokes shift between the original excitation frequency and the final emission frequency. The up-converting phosphors may be organic and inorganic up-converting phosphors. The invention preferably employs up-converting inorganic lanthanide phosphors as labels. Thus, a typical label of the invention is a submicron-size, up-converting lanthanide phosphor particle. The label can alternatively comprise a lanthanide ion in a chelate or cage compound. The up-converting labels of the invention may be attached to a catalyst by adsorbing the catalyst or using various linkage chemistries, as are known to those of ordinary skill in the art.

In general, supports, such as silica or magnesium chloride, may be precipitated from solution to surround the phosphor particle. Various inorganic phosphor coating techniques may be employed including, but not limited to: spray drying, plasma deposition, and derivatization with functional groups (e.g., —COOH, —NH₂, —CONH₂) attached by a silane coupling agent to —SiOH moieties coated on the phosphor particle or incorporated into a phosphor particle comprising silicon oxide(s) and up-converting phosphor compositions. In a preferred embodiment of the invention, the phosphor labels are typically in the form of beads, coated with a thin layer of silica.

The phosphor beads are then either attached directly to the catalyst or incorporated into a solid support material, which may be attached to the catalyst. Catalysts may be directly attached to the phosphor by covalent linkage, for example through siloxane bonds or through carbon-carbon bonds to linking molecules (e.g., organofunctional silylating agents) that are covalently bonded to or adsorbed to the surface of a phosphor particle.

The molar amount of catalyst and up-converting phosphor label generally depends upon the particular catalyst and which reaction conditions are used. Due to their high sensitivity, large amounts of the up-converting phosphor are generally not necessary to give the desired labeling effect. The catalyst may be adsorbed or otherwise attached to the up-converting phosphor, as discussed. Or, the up-converting phosphor label may be incorporated into the catalyst particle. Another alternative, for supported catalysts, is to incorporate the up-converting phosphor into the support material.

Microcrystalline up-converting phosphor particles are typically smaller than about 7 microns in diameter, preferably less than about 1 micron in diameter (i.e., submicron), and more preferably are about 0.1 to about 0.4 microns or less in diameter. It is generally most preferred that the phosphor particles are as small as possible while retaining sufficient quantum conversion efficiency to produce a detectable signal; however, for any particular application, the selection of the size of the phosphor particle(s) to be used should be at the discretion of the practitioner. For instance, some applications may require a highly sensitive phosphor label that need not be small but must have high conversion efficiency and/or absorption cross-section, while other applications may require a very small phosphor particle, but which need not have high conversion efficiency. Therefore, the optimal size of inorganic phosphor particle is application dependent and is usually selected by the practitioner on the basis of quantum efficiency data for various phosphors. Such conversion efficiency data may be obtained from available sources (e.g., handbooks and published references) or may be obtained by generating a standardization curve measuring quantum conversion efficiency as a function of particle size. In some applications, such as those requiring highly sensitive detection of small phosphor particles, infrared laser diodes are preferably selected as an excitation source.

Although the properties of the up-converting phosphors are described in detail in a later section, it is useful to outline the basic mechanisms involved. Up-conversion has been found to occur in certain materials containing rare earth ions in certain crystal materials. For example, ytterbium and erbium act as an activator couple in the phosphor host material barium-yttrium-fluoride. The ytterbium ions act as the absorber and transfer energy non-radiatively to excite the erbium ions. The emission is thus characteristic of the erbium ion's energy levels.

### UP-CONVERTING PHOSPHORS

The up-converting phosphor label is used in this invention to uniquely identify a particular catalyst. Individual phosphors, or combinations of phosphors may also be used to uniquely identify a catalyst. The labels may be identified by illumination with an excitation radiation, followed by detection of the emission radiation. Since up-converting phosphors may be differentiated on the basis of the excitation and/or emission wavelength spectra, up-converting phosphors may be used to detect and discriminate multiple catalysts. Different phosphors may be distinguished according to any one of the following embodiments:

(1) In one embodiment, the wavelength of the excitation radiation of a first phosphor is about equal to the wavelength of the excitation radiation of a second phosphor, and the wavelength of the emission radiation of the first phosphor is different from the wavelength of the emission radiation of the second phosphor.

(2) In a second embodiment, the wavelength of the excitation radiation of a first phosphor is different from the wavelength of the excitation radiation of a second phosphor, and the wavelength of the emission radiation of the first phosphor is about the same as the wavelength of the emission radiation of the second phosphor.

(3) In a third embodiment, the wavelength of the excitation radiation of a first phosphor is different from the wavelength of the excitation radiation of a second phosphor, and the wavelength of the emission radiation of the first phosphor is different from the wavelength of the emission radiation of the second phosphor.

For example, two different catalysts may be attached to two different phosphors (for illustration, designated here as Phosphor#1 and Phosphor#2) which differ in their absorption and/or emission spectra so as to facilitate discrimination of the two phosphors based on absorption and/or emission wavelengths; e.g., one phosphor may emit in the blue and the other may emit in the green. For example Na(Y_{0.80} Yb_{0.18} Er_{0.02})F₄ emits predominantly in the green, and Na(Y_{0.73} Yb_{0.27}Tm_{0.001})F₄ emits predominantly in the blue. Thus these two phosphors may be discriminated on the basis of their phosphorescent emissions. Alternatively, two phosphors may produce essentially similar emission spectra but may have different excitation wavelengths, which provide a basis for their discrimination in multiple catalyst detection and identification.

An up-converting phosphor where the excitation radiation is in the near infrared and the emission radiation is in the visible is preferably used in this invention. A particular advantage of up-converting phosphors is the elimination of background fluorescence or phosphorescence due to the polymer or other material present in the reaction. The phosphors up-convert infrared light for excitation to visible light for detection while other materials typically down-convert and radiate in the UV region. Although the invention can be practiced with a variety of up-converting inorganic phosphors, it is believed that the preferred embodiment(s) employ one or more phosphors derived from one of several different phosphor host materials, each doped with at least one activator couple. Suitable phosphor host materials include: sodium yttrium fluoride (NaYF₄), lanthanum fluoride (LaF₃), lanthanum oxysulfide, yttrium oxysulfide, yttrium fluoride (YF₃), yttrium gallate, yttrium aluminum garnet, gadolinium fluoride (GdF₃), barium yttrium fluoride (BaYF₅, BaY₂F₈), gadolinium oxysulfide, alumina, and aluminates. In addition to the typical phosphors, various ferrite core or supermagnetic materials may be used for magnetic labeling and/or separation techniques. Use of magnetic cores allows for additional labeling and separation using the same up-converting phosphor labels with magnetic and non-magnetic cores while requiring excitation and detection apparatus for a single group of up-converting phosphors.

Suitable activator couples include: ytterbium/erbium, ytterbium/thulium, and ytterbium/holmium. Other activator couples known to those of skill in the art and suitable for up-conversion may also be used. By combining these host materials with the activator couples, at least three phosphors with at least three different emission spectra (red, green, and blue visible light) are generated.

Generally, the absorber is ytterbium and the emitting center is erbium, holmium, terbium, or thulium; however, other up-converting phosphors of the invention may contain other absorbers and/or emitters. The molar ratio of absorber:emitting center is typically at least about 1:1, preferably about 3:1 to 250:1, more preferably about 8:1 to 100:1, and most preferably about 11:1 to 50:1, although various ratios may be selected by the practitioner on the basis of desired characteristics (e.g., chemical properties, manufacturing efficiency, absorption cross-section, excitation and emission wavelengths, quantum efficiency, or other considerations). The ratio(s) chosen will generally also depend upon the particular absorber-emitter couple(s) selected, and may be calculated from reference values in accordance with the desired characteristics.

The optimum ratio of absorber (e.g., ytterbium) to emitting center (e.g., erbium, thulium, or holmium) varies, depending upon the specific absorber/emitter couple. For example, the absorber:emitter ratio for Yb:Er couples is typically in the range of about 20:1 to about 100:1, whereas the absorber:emitter ratio for Yb:Tm and Yb:Ho couples is typically in the range of about 500:1 to about 2000:1. These different ratios are attributable to the different matching energy levels of the Er, Tm, or Ho with respect to the Yb level in the crystal. For most applications, up-converting phosphors may conveniently comprise about 10-30% Yb and (a) about 1-2% Er; (b) about 0.1-0.05% Ho; or (c) about 0.1-0.05% Tm, although other formulations may be employed.

It is preferable to employ Inorganic up-converting phosphors that are optimally excited by infrared radiation at wavelengths of about 950 to 1000 nm, preferably at about 960 to 980 nm. For example, a microcrystalline inorganic phosphor of the formula YF₃:Yb_{0.10} Er_{0.01} exhibits a luminescence intensity maximum at an excitation wavelength of about 980 nm. Inorganic phosphors of the invention typically have emission maxima that are in the visible range. For example, specific activator couples have characteristic emission spectra: ytterbium-erbium couples have emission maxima in the red or green portions of the visible spectrum, depending upon the phosphor host; ytterbium-holmium couples generally emit maximally in the green portion, ytterbium-thulium typically have an emission maximum in the blue range; and ytterbium-terbium usually emit maximally in the green range. For example, Y_{0.80}Yb_{0.19}Er_{0.01}F₂, a ytterbium-erbium copule, emits maximally in the green portion of the spectrum.

Although up-converting inorganic phosphor crystals of various formulae are suitable for use in the invention, the following formulae are generally suitable:

Na(YₓYb_{y}Er_{z})F₄: wherein x is 0.7 to 0.9, y is 0.09 to 0.29, and z is 0.05 to 0.01;

Na(YₓYb_{y}Ho_{z})F₄: wherein x is 0.7 to 0.9, y is 0.0995 to 0.2995, and z is 0.0005 to 0.001;

Na(YₓYb_{y}Pr_{z})F₄: wherein x is 0.7 to 0.9, y is 0.0995 to 0.2995, and z is 0.0005 to 0.001;

Na(YₓYb_{y}Tm_{z})F₄: wherein x is 0.7 to 0.9, y is 0.0995 to 0.2995, and z is 0.0005 to 0.001; and

(YₓYb_{y}Er_{z})O₂S: wherein x is 0.7 to 0.9, y is 0.05 to 0.12; z is 0.05 to 0.12.

(Y_{0.86}Yb_{0.08}Er_{0.06})₂O₃ is a relatively efficient up-converting phosphor material.

To exemplify, ytterbium(Yb)-erbium(Er)-doped yttrium oxysulfides luminesce in the green after excitation at 950 nm. These are non-linear phosphors, in which the ytterbium acts as an “antenna” (absorber) for two 950 nm photons and transfers its energy to erbium, which acts as an emitter (activator). The critical grain size of the phosphor is given by the quantum yield for green emission and the doping level of both Yb and Er, which is preferably in the range of about 1 to 10 percent, more preferably in the range of about 2 to 5 percent. A typical Yb:Er phosphor crystal comprises about 10-30% Yb and about 1-2% Er. Thus, a phosphor grain containing several thousand formula units ensures the emission of at least one or more photons during a typical laser irradiation time. However, the nonlinear relationship between absorption and emission indicates that intense illumination at the excitation wavelength(s) may be necessary to obtain a satisfactory signal in embodiments employing very small phosphor particles (i.e., less than about 0.3 μm). Additionally, it is usually desirable to increase the doping levels of activator/emitter couples for producing very small phosphor particles so as to maximize quantum conversion efficiency.

Inorganic microcrystalline up-converting phosphors with rare earth activators generally have narrow absorption and line emission spectra. The line emission spectra are due to ƒ-ƒ transitions within the rare earth ion. These are shielded internal transitions, which result in narrow line emission.

In certain applications, such as where highly sensitive detection is required, intense illumination may be provided by commercially available sources, such as infrared laser sources (e.g., continuous wave (CW) or pulsed semiconductor laser diodes). For example, in applications where the microcrystalline phosphor particle must be very small and the quantum conversion efficiency is low, intense laser illumination can increase signal and decrease detection times. Alternatively, some applications of the invention may require phosphor compositions that have inherently low quantum conversion efficiencies (e.g., low doping levels of activator couple), but which have other desirable characteristics (e.g., manufacturing efficiency, ease of derivatization, etc.); such low efficiency up-converting phosphors are preferably excited with laser illumination at a frequency at or near (i.e., within about 25 to 75 nm) an absorption maximum of the material. The fact that no other light is generated in the system other than from the up-converting phosphor allows for extremely sensitive signal detection, particularly when intense laser illumination is used as the source of excitation radiation. This unique property of up-conversion of photon energy by up-converting phosphors makes possible the detection of very small particles of microcrystalline inorganic phosphors. For practical implementation of up-converting phosphors as ultrasensitive labels, the grain size of the phosphor should be as small as practicable (typically less than about 0.3 to about 0.1 μm), for which laser-excited up-converting phosphors are well-suited.

For example, various phosphor material compositions capable of up-conversion are suitable for use in the invention are shown in Table I.

TABLE I
Phosphor Material Compositions

| Host Material | Absorber Ion | Emitter Ion | Color |
|---|---|---|---|
| Oxysulfides (O₂S) | | | |
| Y₂O₂S | Ytterbium | Erbium | Green |
| Gd₂O₂S | Ytterbium | Erbium | Red |
| La₂O₂S | Ytterbium | Holmium | Green |
| Oxyhalides (OX_{y}) | | | |
| YOF | Ytterbium | Thulium | Blue |
| Y₃OCI₇ | Ytterbium | Terbium | Green |
| Fluorides (Fₓ) | | | |
| YF₃ | Ytterbium | Erbium | Red |
| GdF₃ | Ytterbium | Erbium | Green |
| LaF₃ | Ytterbium | Holmium | Green |
| NaYF₃ | Ytterbium | Thulium | Blue |
| BaYF₅ | Ytterbium | Thulium | Blue |
| BaY₂F₆ | Ytterbium | Terbium | Green |
| Gallates (GaₓO_{y}) | | | |
| YGaO₃ | Ytterbium | Erbium | Red |
| Y₃Ga₅O₁₂ | Ytterbium | Erbium | Green |
| Silicates (SiₓO_{y}) | | | |
| YSi₂O₅ | Ytterbium | Holmium | Green |
| YSi₃O₇ | Ytterbium | Thulium | Blue |

In addition to the materials shown in Table I and variations thereof, aluminates, phosphates, and vanadates may be used as suitable phosphor host materials. In general, when silicates are used as a host material, the conversion efficiency is relatively low. In certain uses, hybrid up-converting phosphor crystals may be made (e.g., combining one or more host material and/or one or more absorber ion and/or one or more emitter ion).

Exemplary up-converting phosphors excited at about 980 nm include, but are not limited to: (Y_{0.80}Yb_{0.18}; Er_{0.02})F₃; (Y_{0.87}Yb_{0.13}Tm_{0.001})F₃ ; (Y_{0.80}Yb_{0.198}Ho_{0.002})F₃; (Gd_{0.80}Yb_{0.18}Er_{0.02})F₃; (Gd_{0.87}Yb_{0.13}Tm_{0.001})F₃; (Gd_{0.80}Yb_{0.198}Ho_{0.002})F₃; (Y_{0.86}Yb_{0.08}Er_{0.06})₂ O₂ S; (Y_{0.87}Yb_{0.13}Tm_{0.001})₂ O₂ S; (Y_{0.80}Yb_{0.198}Ho_{0.002})₂ O₂ S; (Gd_{0.86}Yb_{0.08}Er_{0.06})₂ O₂ S; (Gd_{0.87}Yb_{0.13}Tm_{0.001})₂ O₂ S; and (Gd_{0.80}Yb_{0.198}Ho_{0.002})₂ O₂ S. Exemplary up-converting phosphors excited at about 1500 nm include, but are not limited to: (Y_{0.96}Er_{0.06})₂O₂ S; (Gd_{0.96}Er_{0.06})₂ O₂ S.

Preparation of Inorganic Up-Converting Phosphors

Techniques and methods for manufacture of inorganic phosphors has been described in the art. Up-converting phosphor crystals may be manufactured according to various published methods, including but not limited to the following: Yocom et al., Metallurgical Transactions 2:763 (1971); Kano et al., J. Electrochem. Soc., p. 1561 (1972); Wittke et. al., J. Appl. Physics 43:595 (1972); Van Uitert et al., Mat. Res. Bull. 4:381 (1969); which are incorporated herein by reference. Other references that may be referred to are: Jouart J. P. and Mary G, J. Luminescence 46:39 (1990); McPherson G. L. and Meyerson S. L., Chem. Phys. Lett. (April) p. 325 (1991); Oomen et al., J. Luminescence 46:353 (1990); NIH and Rand S. C., Optics Lett. 16 (September) (1991); McFarlane R. A., Optics Lett. 16 (September) (1991); Koch et al., Appl. Phys. Lett. 56:1083 (1990); Silversmith et al., Appl Phys. Lett. 51:1977 (1987); Lenth W and McFarlane R. M., J. Luminescence 45:346 (1990); Hirao et al., J. Non-crystalline Solids 135:90 (1991); McFarlane et al., Appl. Phys. Lett. 52:1300 (1988), which are all incorporated herein by reference.

The techniques required to incorporate the phosphors into catalyst and catalyst support particles vary widely and are dependent on the desired catalyst. Leading references can be found in Hlatky, G. G., “Heterogeneous Single-Site Catalysts for Olefin Polymerization”, Chemical Reviews, 100:1347-1376 (2000); and Roscoe, Stephen B., Frechet, Jean M. J., Walzer, John F., Dias, Anthony J., “Polyolefin Spheres from Metallocenes Supported on Noninteracting Polystyrene”, Science, 280:270-273 (1998). In an embodiment of the invention, the up-converting phosphors are present as beads, which are used in a variety of combinations, to identify a particular catalyst. In a preferred embodiment of the invention, the phosphor bead is about 0.4 μm in diameter and coated with a thin layer of silica. The phosphors are incorporated into individual catalyst or support particles (e.g. using precipitation techniques). In a preferred embodiment, about 10 phosphors are incorporated into each labeled catalyst particle for detection purposes. The variety of distinct labels are increased by varying the number and type of phosphors incorporated within each support particle. In a typical embodiment, the labeled catalyst particles will preferably be about 5 μm to incorporate a sufficient number of phosphors.

In general, inorganic phosphor particles are milled to a desired average particle size and distribution by conventional milling methods known in the art, including milling in a conventional barrel mill with zirconia and/or alumina balls for periods of up to about 48 hours or longer. Phosphor particles used are preferably about 3.0 μm to about 0.01 μm in diameter (or along the long axis if non-spherical), more preferably about 2.0 μm to about 0.1 μm in diameter, and most preferably about 1.0 μm to about 0.3 μm in diameter, although phosphor particles larger or smaller than these dimensions may be preferred for certain embodiments. Phosphor particle size is selected by the practitioner on the basis of the desired characteristics and in accordance with the guidelines provided herein. Fractions having a particular particle size range may be prepared by sedimentation, generally over an extended period (i.e., a day or more) with removal of the desired size range fraction conducted after the appropriate sedimentation time. The sedimentation process may be monitored, with any monitoring device known in the art, such as a Horiba Particle Analyzer.

Milling crystalline materials has several weaknesses. With milling, the particle morphology is not uniform, as milled particles result from random fracture of larger crystalline particles. Since the sensitivity of a detection assay using up-converting inorganic phosphors depends on the ability to distinguish between bound and unbound phosphor particles, it is preferable that the particles be of identical size and morphology. Size, weight, and morphology of up-converting microcrystalline phosphor particles may affect the number of potential binding sites per particle and thus the potential strength of particle binding to the phosphor. Monodisperse submicron spherical particles of uniform size may be generated by homogeneous precipitation reactions at high dilutions. For example, small yttrium hydroxy carbonate particles are formed by the hydrolysis of urea in a dilute yttrium solution. Similarly, up-converting inorganic phosphors may be prepared by homogeneous precipitation reactions in dilute conditions. For example, (Y_{0.86}Yb_{0.08}Er_{0.06})₂ O₃ was prepared as monodisperse spherical particles in the submicron size range by precipitation.

However, after precipitation, it is typically necessary to anneal the oxide in air at about 1500° C., which may cause faceting of the spherical particles, which in turn, may generate aggregate formation. Faceting can be substantially reduced by converting the small spherical particles of the oxide or hydroxy carbonate precursor to the oxysulfide, fluroide, or other desired host phase by a fluidized bed process. Using this technique, highly efficient oxysulfide and fluoride particles in the 0.3 to 0.4 μm diameter range may be prepared. See U.S. Pat. No. 6,039,894, which is hereby incorporated by reference. Frequently, sonication can be used to produce a monodisperse mixture of discrete spherical particles. After fractionation and coating, these particles can be used as up-converting phosphors. Furthermore, this general preparative procedure is suitable for preparing much smaller phosphor particles (e.g., 0.1 μm diameter or smaller), which may be advantageous for various assay formats.

Frequently, such as with phosphors having an oxysulfide host material, the phosphor particles are preferably dispersed in a polar solvent, such as acetone or DMSO and the like, to generate a substantially monodisperse emulsion (e.g., for use in a stock solution). Aliquots of the monodisperse stock solution may be further diluted into an aqueous solution (e.g., a solution of avidin in buffered water or buffered saline). Washing phosphors in acetone or DMSO often improves suspendability of inorganic phosphor particles in water. In particular, the phosphor particles prepared with polysulfide flux are preferably resuspended and washed in hot DMSO and heated for about an hour in a steam bath then allowed to cool to room temperature under continuous agitation. The phosphor particles may be pre-washed with acetone (typically heated to boiling) prior to placing the particles in the DMSO. Hot DMSO-treated phosphors were found to be reasonably hydrophilic and form stable suspensions. A Microfluidizer™ (Microfluidics Corp.) may be used to further improve the dispersion of particles in the mixture. DMSO-phosphor suspensions may be mixed with water, preferably with mall amounts of surfactant present. In general, polysaccharides (e.g., guar gum, xanthan gum, gum arabic, lignate, guaiac gum) may be used to promote deaggregation of particles. Additionally, particles may be washed in hot DMSO and serially diluted into a 0.1% aqueous gum arabic solution, to eliminate much of the water dispersion problems associated with phosphors.

Resuspended phosphors in organic solvents, such as DMSO, are typically allowed to settle for a suitable period (e.g., about 1-3 days), and the supernatants which is typically turbids is used for subsequent conjugation.

Tetraethylorthosilicate (TEOS) in 95% ethanol/water may be used to coat up-converting phosphor particles, which can be subsequently heated to form a ceramic silica coating that cannot be removed from the phosphor particles, but which can be readily silanized with organofunctional silanes (containing thiol, primary amine, and carboxylic acid functionalities) using standard silanization chemistries (See Arkles, B., Silicon Compounds: Register and Review, 5th Edition (1991); Anderson, R. G., Larson, G. L., and Smith, C., eds., p. 59-64, Huls America, Piscataway, N.J.).

Phosphor particles can be coated or treated with surface-active agents (e.g., anionic surfactants such as Aerosol OT) during the milling process or after milling is completed. For example, particles may be coated with a polycarboxylic acid (e.g., Additon XW 330, Hoechst, Frankfurt, Germany or Tamol, see Beverloo et al. (1992) op.cit.) during milling to produce a stable aqueous suspension of phosphor particles, typically at about pH 6-8. The pH of an aqueous solution of phosphor particles can be adjusted by addition of a suitable buffer and titration with acid or base to the desired pH range. Depending upon the chemical nature of the coating, some minor loss in conversion efficiency of the phosphor may occur as a result of coating, however the power available in a laser excitation source can compensate for such reduction in conversion efficiency and ensure adequate phosphor emission.

In general, preparation of inorganic phosphor particles and linkage to biological binding reagents is described in Beverloo et al. (1992) op.cit., and Tanke U.S. Pat. No. 5,043,265. Alternatively, a water-insoluble polyfunctional polymer that exhibits glass and melt transition temperatures well above room temperature may be used to coat the up-converting phosphors in a nonaqueous medium. For example, such polymer functionalities include: carboxylic acids (e.g., 5% acrylic acid/95% methyl acrylate copolymer), amine (e.g., 5% aminoethyl acrylate/95% methyl acrylate copolymer) reducible sulfonates (e.g., 5% sulfonated polystyrene), and aldehydes (e.g., polysaccharide copolymers). The phosphor particles are coated with water-insoluble polyfunctional polymers by coacervative encapsulation in nonaqueous media, washed, and transferred to a suitable aqueous buffer solution to conduct the crosslinking to a support or catalyst particle. An advantage of using water-insoluble polymers is that the polymer microcapsule will not migrate from the surface of the phosphor upon aging the encapsulated phosphors in an aqueous solution (i. e., improved reagent stability). Since the solubility and coacervative encapsulation process will depend on the dominant nonfunctionalized component of the copolymer, the functionalized copolymer ratio can be varied over a wide range to generate a range of potential crosslinking sites per phosphor, without having to substantially change the encapsulation process.

Methods of Using the Labeled Catalyst in Synthetic Reactions

Once the labeled catalyst particle has been prepared, it is then possible to use the labeled catalyst particle in a variety of catalytic reactions. Examples of typical catalytic reactions/transformations include, but are not limited to, polymerizations, oxidations, reductions, hydrosilation, carbonylations, hydrocynation, hydroformylation, isomerization, metathesis, carbon-hydrogen activation, cross coupling, Friedel-Crafts acylation and alkylation, hydration, Diels-Alder reactions, condensation, decomposition, hydrogenation, hydrosilylation, hydrocyanation, carbonylation, cross coupling, epoxidation, dimerization, hydroboration, hydrohalogenation, hydroxylation, halohydrination, alkylation, carbene addition, dihalo carbene addition, and combinations of these.

Catalyst activity may be determined by measuring a property or parameter of the reaction and then using the label of the labeled catalyst particle to attribute or match the measured property to a particular catalyst. For example, a mixture of labeled catalysts may be used as a catalyst bed for a particular reaction. The relative temperature of the bed may be measured and then the labels used to identify the catalyst corresponding to a particular temperature region. Matching the particular catalyst to the temperature parameter, for example, permits the activity of multiple catalysts to be studied.

In a preferred embodiment, the labeled catalyst particle is used in a polymerization method. Labeled catalyst particles are mixed together in a polymerization reactor and contacted with a monomer or mixture of co-monomers to provide a desired polymer. Polymerization of the monomer (or co-monomers) produces a polymer that results in the formation of a non-aggregating polymer bead around the labeled catalyst particle. The catalyst particles produce a single polymer bead, which maintains its up-converting phosphor label. Any undesired amorphous polymer may be discarded.

More particularly, this method comprises the step of first combining a monomer (or mixture of co-monomers) with at least one labeled catalyst particle, wherein the labeled catalyst particle comprises at least one up-converting phosphor label and a catalyst, and optionally a co-catalyst. As described above, the up-converting phosphor label identifies a particular catalyst, and comprises at least one rare earth ion and at least one phosphor host material, as well as a support material.

The monomer may then be polymerized to form a polymer bead surrounding the labeled catalyst particle. In general, each labeled catalyst particle must form one polymer particle or polymer bead, and the particles should preferably not stick together, (i.e. the polymer particles should be non-aggregating) in order to facilitate sorting the resulting products.

In yet another embodiment, the invention relates to polymerization reactions producing either crystalline polymers, or amorphous polymers. As an example, in a staged reactor process, a hard, porous polymer is formed and then passed to a second reactor where a soft, sticky polymer forms within the pores. For a general discussion of this process, see U.S. Pat. Nos. 6,028,140 and 5,648,422, as well as Galli, P. et al., “Combining Ziegler-Natta and Metallocene Catalysis: New Heterophasic Propylene Copolymers from the Novel Multicatalyst Reactor Granule Technology,” Journal of Applied Polymer Science, 66:1831-1837 (1997), which are all hereby incorporated by reference.

As described in greater detail in the description that follows, the polymer beads retain phosphor labels that identify the catalysts. The phosphor can be detected by illuminating the polymer beads with excitation radiation, and then detecting the emission radiation of one or more wavelengths. The resulting polymer beads can then be sorted, for example, based on size and/or by phosphor label, e.g., using a modified commercial cytometer. The resulting polymer beads may also be further characterized for various polymer properties and characteristics.

The catalyst used in the labeled catalyst particle may be any chemical compound that has the ability to accelerate or initiate chemical reactions, without itself being consuned or undergoing a chemical change in the overall reaction. Typical catalysts include inorganic compounds, organic compounds, and complexes of organic groups and metal halides. The catalyst may be selected from a wide variety of catalysts including, but not limited to, mixed metal oxides, mixed metal nitrides, mixed metal sulfides, mixed metal carbides, mixed metal fluorides, mixed metal silicates, mixed metal aluminates, mixed metal phosphates, noble metals, zeolites, metal alloys, intermetallic compounds, inorganic mixtures, inorganic compounds, inorganic salts, radical catalysts, cationic catalysts, anionic catalysts, anionic coordination catalysts, and mixtures thereof. See Richard J. Lewis, Sr., Hawley's Condensed Chemical Dictionary, Thirteenth Edition, John Wiley & Sons, Inc., New York, 1997. Typical catalysts, which may be used in the invention, include but are not limited to: Zieglar-Natta catalysts, metallocene catalysts, stereospecific catalysts, constrained geometry catalysts, single-site catalysts, late transition metal single-site catalysts, free radial initiators, living free radical initiators, cationic initiators, anionic initiators, co-ordination complexes and mixtures thereof.

In a preferred embodiment, the catalysts are supported catalysts. In general, the solid support material consists of any material known to one of ordinary skill in the art. Typical supported catalyst, and methods of making and using precipitated catalyst supports in polymerization processes are known to those of ordinary skill in the art, and are described, for example, in U.S. Pat. Nos. 5,747,407; 5,206,314; 5,081,090; 4,946,816; 4,831,091; and 4,567,155, and WO 92/13009, as well as George M. Benedikt and Brian L. Goodall, Eds., Metallocene-Catalyzed Polymers: Materials, Properties, Processing & Markets, Plastics Design Library, (1996); George M. Benedikt and Brian L. Goodall, Eds., Metallocene Technology: in Commercial Applications, Plastic Design Library, William Andrew Inc. (1999); Fink, G.; Muelhaupt, R.; Brintzinger, H. H.; Editors; Zieglar Catalysis, Springer, Berlin, Germany, (1995); Peter Roos et al., Macromol. Rapid Commun., 18:319-324 (1997); and Elodie Bourgeat-Lamie and Jacques Lang, Journal of Colloid and Interface Science, 197:293-308 (1998), which are all hereby incorporated by reference.

Typical solid support materials, include but are not limited to: porous resinous materials, such as copolymers of styrene-divinylbenzene, or solid inorganic oxides, such as silica, alumina, magnesium oxide, titanium oxide, thorium oxide, mixed oxides of silica, and one or more Group 2 or 13 metal oxides. Preferably, the solid support material a silica-magnesia mixed oxide, a silica-alumina mixed oxide, silica, alumina, or Group 2 or 13 metal oxide.

The polymerization reaction may be carried out under suitable reaction conditions as are known to those of ordinary skill in the art. In addition to the labeled catalyst particle, the polymerization reaction may typically require initiators, activators, heat and/or light. The polymerization reaction may be run under high pressure. The polymerization reaction may also be carried out (1) in the gas phase, typically at high pressures and temperatures of about 200° C., (2) in solution at normal pressure and temperatures, typically from about −70° C. to about 70° C., (3) bulk or batch polymerization, typically at normal pressures and at temperatures of about 150° C., (4) in suspension at normal pressures, and at temperatures from about 60° C. to about 80° C., or (5) in emulsion form, typically at normal pressures, and at temperatures from about −20 to about 60° C. See Richard J. Lewis, Sr., Hawley's Condensed Chemical Dictionary, Thirteenth Edition, John Wiley & Sons, Inc., New York, 1997.

The polymerization reaction may be carried out in any scale, from microscale to large scale, and may also be carried out in any commercially relevant reactor. This is an advantage over typical combinatorial methods, which often require that reactions be run on the microscale, and/or in expensive apparatus designed for combinatorial synthesis. Typical reactor, include, but are not limited to: a batch reactor, a continuous reactor, a semicontinuous reactor, a gas phase reactor, a fluidized bed reactor, a stirred bed reactor, a slurry phase reactor, an autoclave reactor, an emulsion reactor, a multiwell parallel reactor, multiple reactors in series, a staged reactor, and a cascade reactor.

The invention covers polymerization reactions comprising a variety of monomers, including but not limited to: ethylene, propylene, cis-2-butene, butadiene, 1-hexene, 1-octene, 1-butene, 3-methyl-1-butene, 1,3-butadiene, 1-pentene, 4-methyl-1-pentene, 1-hexene, 4-methyl-1-hexene, 1,4-hexadiene, 1,5-hexadiene, 1-octene, 1,6-octadiene, 1-nonene, 1-decene, 1,4-dodecadiene, 1-hexadecene, 1-octadecene, cyclopentene, 3-vinylcyclohexene, 5-vinyl-2-norbomene, 5-ethylidene-2-norbomene, dicyclopentadiene, 4-vinylbenzocyclobutane, tetracyclododecene, dimethano-octahydronaphthalene, 7-octenyl-9-borabicyclo-(3,3,1)nonane, styrene, o-methylstyrene, m-methylstyrene, p-methylstyrene, p-tert-butylstyrene, m-chlorostyrene, p-chlorostyrene, p-fluorostyrene, indene, 4-vinylbiphenyl, acenaphthalene, vinylfluorene, vinylanthracene, vinylphenanthrene, vinylpyrene, vinylchrisene, methylmethacrylate, ethylacrylate, vinyl silane, phenyl silane, trimethylallyl silane, acrylonitrile, maleimide, vinyl chloride, vinylidene chloride, tetrafluoroethylene, isoprene, isobutylene, carbon monoxide, acrylic acid, 2-ethylhexylacrylate, methyl acrylate, methyl methacrylate, methacrylonitrile, methacrylic acid, vinyl acetate, norbomene, norbomadiene, and mixtures thereof.

In one embodiment of the invention, one or more monomers are polymerized in a polymerization reaction, to form a polymer bead surrounding the labeled catalyst particle. The polymer bead retains the phosphor label. The resulting polymer beads may then be sorted, for example, based on their size, or based upon the phosphor label. A schematic representation of the up-converting phosphor bead based polymerization catalyst screen is shown in FIG. 1. As shown in FIG. 1, up-converting phosphor labeled catalyst particles produce individually labeled polymer beads. The catalyst activity can be determined from the polymer bead size or weight. The polymer beads can be sorted by label, by size, or by label and size. The sorted polymer beads may be further analyzed using conventional or high-throughput techniques.

Typically, the labeled polymer beads are analyzed with a modified commercial cytometer, as is explained in greater detail below. Typically, the size of the polymer beads analyzed by cytometry are about 5 μm to about 5000 μm, preferably about 30 μm to about 500 μm and most preferably about 3001 μm. The cytometer will provide spectral (label identification) and size (proportional to catalyst activity) information for each bead. The beads may then be sorted and analyzed by combinatorial or traditional polymer characterization techniques. In general, it should be noted that the polymer beads can be sorted by size or by bulk density, since the polymers scatter light depending on both size and bulk density.

In addition, the resulting polymer beads, can further be characterized for a number of different polymer properties and characteristics. An advantage of the invention is that the phosphor labels are chemically and thermally compatible with a variety of different assays and characterization methods, such that there is generally no need to cleave or separate the phosphor label from the polymer bead in order to carry out further characterization of the resulting polymer. Examples of properties of the resulting polymer that may be characterized include, but are not limited to: chemical or biological testing, mass spectrometry, viscosity measurement, thermogravimetric analysis (TGA), digital autoradiography, thermal imaging, infrared spectroscopy, reflectance spectroscopy, uv-vis spectroscopy, uv-vis fluorescence, NMR spectroscopy, gas chromatography, high performance liquid chromatography (HPLC), gel permeation chromatography (GPC), temperature rising elution fractionization (TREF), x-ray diffraction, electron spectroscopy, scanning electron microscopy (SEM), transmitting electron microscopy (TEM), scanning tunneling microscopy (STM) and atomic force microscopy (AFM). These techniques may be used alone, or in any combination. Other chemical and physical properties which may be measured, include but are not limited to: solubility, hexane extractables, weatherability, uv-vis stability, scratch resistance, abrasion resistance, wetability, hardness, color, moisture absorption, drying rate, solvent swelling, adhesion, heat aging, shear, stain resistance, color fastness, and scrub resistance.

Detection Apparatus

Detection and quantification of inorganic up-converting phosphor(s) is generally accomplished by: (1) illuminating a sample suspected of containing up-converting phosphors with electromagnetic radiation at an excitation wavelength, and (2) detecting phosphorescent radiation at one or more emission wavelength band(s).

Illumination of the sample is produced by exposing the sample to electromagnetic radiation produced by at least one excitation source. Various excitation sources may be used, including infrared laser diodes and incandescent filaments, as well as other suitable sources. Optical filters which have high transmissibility in the excitation wavelength range(s) and low transmissibility in one or more undesirable wavelength band(s) may be employed to filter out undesirable wavelengths from the source illumination. Undesirable wavelength ranges generally include those wavelengths that produce detectable sample autofluoresence and/or are within about 25-100 nm of excitation maxima wavelengths and thus potential sources of background noise from scattered excitation illumination. Excitation illumination may also be multiplexed and/or collimated; for example, beams of various discrete frequencies from multiple coherent sources (e.g., lasers) may be collimated and multiplexed using an array of dichroic mirrors. In this way, samples containing multiple phosphor species having different excitation wavelength bands may be illuminated at their excitation frequencies simultaneously. Illumination may be continuous or pulsed, or may combine continuous wave (CW) and pulsed illumination where multiple illumination beams are multiplexed (e.g., a pulsed beam is multiplexed with a CW beam), permitting signal discrimination between phosphorescence induced by the CW source and phosphorescence induced by the pulsed source. This creates the ability to discriminate between multiple phosphor species having similar emission spectra but different excitation spectra. For example but not limitation, commercially available gallium arsenide laser diodes may be used as an illumination source for providing near-infrared light.

The ability to use infrared excitation for stimulating up-converting phosphors provides several advantages. First, inexpensive IR and near-IR diode lasers may be used for sustained high-intensity excitation illumination, particularly in IR wavelength bands which are not absorbed by water. This level of high-intensity illumination would not be suitable for use with conventional labels, such as ordinary fluorescent dyes (e.g., FITC), since high-intensity UV or visible radiation produces extensive photobleaching of the label and, potentially, damage to the sample. The ability to use higher illumination intensities without photobleaching or sample damage translates into larger potential signals, and hence more sensitive assays.

The compatibility of up-converting phosphors labels with the use of diode lasers as illumination sources provide other distinct advantages over lamp sources and most other laser sources. First, diode laser intensity can be modulated directly through modulation of the drive current. This allows modulation of the light for time-gated or phase-sensitive detection techniques, which afford sensitivity enhancement without the use of an additional modulator. Modulators require high-voltage circuitry and expensive crystals, adding both cost and additional size to apparatus. The laser diode or light-emitting diode may be pulsed through direct current modulation. Second, laser illumination sources provide illumination that is exceptionally monochromatic and can be tightly focused on very small spot sizes, which provides advantages in signal-to-noise ratio and sensitivity due to reduced background light outside of the desired excitation spectral region and illuminated volume. A diode laser affords these significant advantages without the additional expense and size of other conventional or laser sources.

Detection and quantification of phosphorescent radiation from excited up-converting phosphors can be accomplished by a variety of means. Various means of detecting phosphorescent emission(s) can be employed, including but not limited to: photomultiplier devices, avalanche photodiode, charge-coupled devices (CCD), CID devices, photographic film emulsion, photochemical reactions yielding detectable products, and visual observation (e.g., fluorescent light microscopy). Detection can employ time-gated and/or frequency-gated light collection for rejection of residual background noise. Time-gated detection is generally desirable, as it provides a method for recording long-lived emission(s) after termination of illumination; thus, signal(s) attributable to phosphorescence or delayed fluorescence of up-converting phosphor is recorded, while short-lived autofluoresence and scattered illumination light, if any, is rejected. Time-gated detection can be produced either by specified periodic mechanical blocking by a rotating blade (i.e., mechanical chopper) or through electronic means wherein prompt signals (i.e., occurring within about 0.1 to 0.3 μs of termination of illumination) are rejected (e.g., an electronic-controlled, solid-state optical shutter such as Pockel's or Kerr cells). Up-converting phosphors and up-converting delayed fluorescent dyes typically have emission lifetimes of approximately a few milliseconds (perhaps as much as 10 ms, but typically on the order of 1 ms), whereas background noise usually decays within about 100 ns. Therefore, when using a pulsed excitation source, it is generally desirable to use time-gated detection to reject prompt signals.

Since up-converting phosphors are not subject to photobleaching, very weak emitted phosphor signals can be collected and integrated over very long detection times (continuous illumination or multiple pulsed illumination) to increase sensitivity of detection. Such time integration can be electronic or chemical (e.g., photographic film). When non-infrared photographic film is used as a means for detecting weak emitted signals, up-converting phosphors provide the advantage as compared to down-converting phosphors that the excitation source(s) typically provide illumination in a wavelength range (e.g., infrared and near infrared) that does not produce significant exposure of the film (i.e., is similar to a darkroom safelight). Thus, up-converting phosphors may be used as convenient ultrasensitive labels for immunohistochemical staining and/or in situ hybridization in conjunction with fluorescence microscopy using an infrared source (e.g., a infrared laser diode) and photographic film (e.g., Kodak Ektachrome) for signal and image detection of visible range luminescence (with or without an infrared-blocking filter).

Instrumentation Overview

The basic purpose of the instrumentation is to expose the up-converting phosphor label to excitation radiation, e.g., near-infrared (NIR) light, and to measure the resulting emission radiation, e.g. visible light that is emitted. While certain embodiments are described below, other methods known in the art may also be used.

Since the excitation is a two-photon process, there is no requirement that the two photons have the same energy. Rather, it is only necessary that the total energy of the two photons fall within the excitation band. Thus, since it is relatively straightforward and inexpensive to provide different wavelengths with laser diodes, there are more possible combinations, i.e., more possible choices of total excitation energy. This allows more latitude in the choice of rare earth ions for up-converters since the excitation steps need not rely on energy transfer coincidences involving a single photon energy. Further, it may be possible to achieve direct stepwise excitation of the emitting ion (the erbium ion in the example outlined above) without using energy transfer from another absorbing ion (the ytterbium ion in the example) while taking advantage of resonant enhancement of intermediate levels. Additionally, the use of different wavelengths for a single phosphor can provide additional options for excitation-dependent multiplexing and background discrimination techniques.

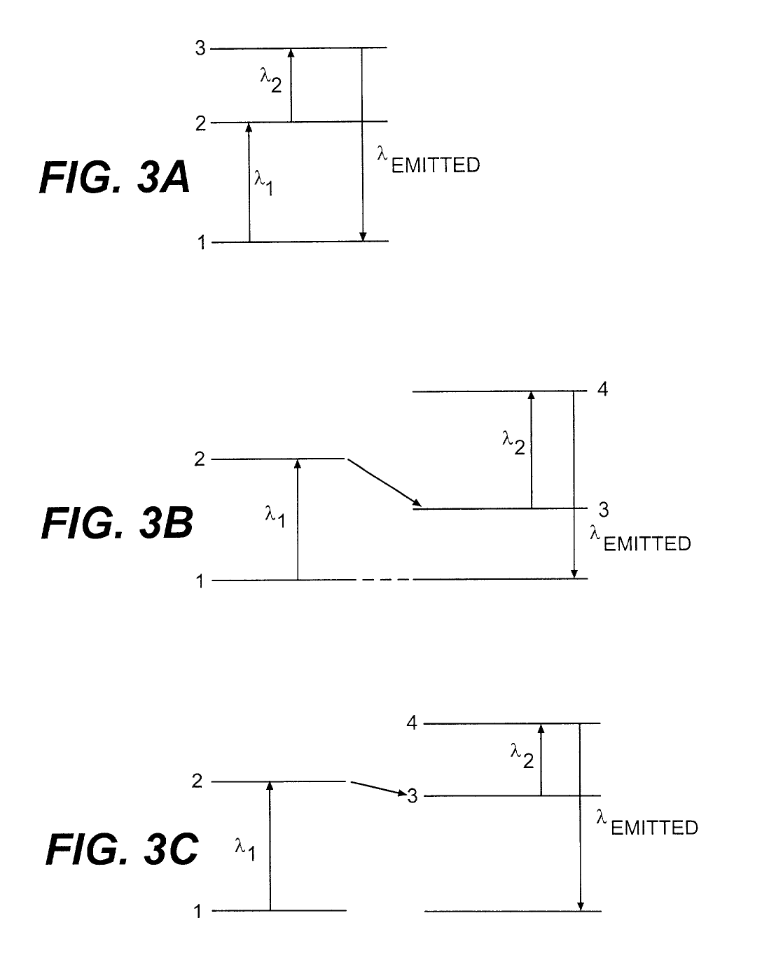

Multiple wavelength excitation of a single phosphor may occur in a number of ways, as shown in FIGS. 3A through 3C. Two lasers may cause stepwise excitation of a single ion, as shown in FIG. 3A. A first laser stimulates excitation from level 1 to level 2, and a second laser stimulates excitation from level 2 to level 3, at which level emission occurs. Single ion excitation can also occur using energy transfer as shown in FIG. 3B. In this case, a first laser stimulates excitation from level 1 to level 2, energy transfer occurs from level 2 to level 3, and a second laser stimulates excitation from level 3 to level 4. In a variation of the latter process, levels 1 and 2 can be in a first ion (i.e., a sensitizer ion) and levels 3 and 4 in a second ion (i.e., activator ion), as shown in FIG. 3C.

In a stepwise excitation scheme shown in FIG. 3A, energy transfer is not required, and thus information on the polarization of the excitation lasers may be preserved and cause polarization of the emitted radiation. In this case, depolarization of the light may allow for enhanced discrimination between signal and background noise.

For the multi-ion multi-laser excitation scheme shown in FIG. 3C, there may be several phosphors that share a common excitation wavelength. In this case, discrimination between different phosphors may be performed on the basis of different emission wavelengths and/or through time-gated, frequency-modulated, and/or phase-sensitive detection utilizing modulation of the excitation wavelength(s).

Phosphor-Based Flow Cytometer

Flow cytometers using up-converting phosphors are described in U.S. Appl. No. 09/488,797, filed on Jan. 21, 2000, and U.S. Provisional Appl. No. 60/116,965 filed Jan. 22, 1999, which are incorporated by reference. The present application incorporates by reference U.S. Pat. Nos. 5,674,698, 5,698,397, and Flow Cytometry with Upconverting Phosphors Reporters” by W. H. Wright et al. in SPIE Proceedings: Optical Investigations of Cells In Vitro and In Vivo, D. L. Farkas et al. (editors), Vol. 3260, published April 1998, pp. 245-254. While it is possible to use the upconverting phosphors for flow cytometry, several equipment design issues related to the unique excitation and emission characteristics of these phosphors must be addressed.

Excitation Source

The first issue is the time required to reach maximum emission intensity (rise time). Up-conversion is a two photon process; that is, the energy must be trapped from the first absorbed photon and pumped by the absorption of a second photon to result in the emission of a single up-converted photon. Because of this phenomena, upconverting phosphor emission is time-delayed about 100 μsec. The phosphor must remain within the beam of the excitation source for at least 100 μsec at all flow rates. Assuming typical flow rates of 1 to 10 m/sec in flow cytometers and channel widths of 70 to 200 μm, the length of the excitation laser beam must be between 100 and 1000 μm. Phosphor emission saturates at an excitation intensity of about 200 W/cm². This means that the excitation source laser can emit at a power of between 0.01 and 400 mW to achieve phosphor saturation.

Detection

Since emission from the phosphors is in the visible spectrum, and the wavelength of the excitation source (980±5 nm) is far removed from that of emission (≧800 nm), high sensitivity detection is easily accomplished with a photomultiplier tube (PMT), photodiode, or a CCD array that is shielded from the excitation beam with a cutoff filter.

There is no need for time resolved measurements when using upconverting phosphors. This means that excitation and detection can be accomplished simultaneously and continuously. Representing a major advantage over conventional time-resolved detection systems. Diode array detectors could also be used to take advantage of the different wavelength emissions of different phosphor compositions. A diode array detector would make it possible to perform multiplexed assays, such as simultaneously accomplishing phosphor bead sorting based on more than one characteristic.

With regard to up-converting phosphors, the phosphorescence decay time is long, at least compared to common UV fluorescent phosphors such as fluorescein. The phosphorescence decay half life is about 300 μsec. The most sensitive method of detection would be to integrate the signal measured by the PMT However, 99 percent detection of the available phosphorescent signal requires that the phosphor remain in the sight path of the detector for 5 times the phosphorescence decay half-life, or 1.5 msec.

It is possible to sacrifice some detection sensitivity by reducing the detection path length, at least to that required to attain steady-state emission from the phosphors—that is, 0.1 cm. As long as a steady-state emission peak is reached by the phosphor in the excitation window, the peak signal received by the PMT should be directly proportional to the concentration of phosphors present. The non-photobleaching property of the phosphors makes this form of detection possible. The loss in detection sensitivity corresponding to a 0.1 cm detection path (vs. 1.5 cm) should only be about a factor of 3. Triggering of the emission detector could be accomplished by observing the light scattered by the phosphor bead as it passes through the excitation laser using a silicon photodiode.

The phosphor-based design for the flow-cytometer provides for both forward scatter detection (particle size information) at 980 nm and phosphorescence detection. Phosphorescence detection uses either the spectroscopic or non-dispersed filtered designs.

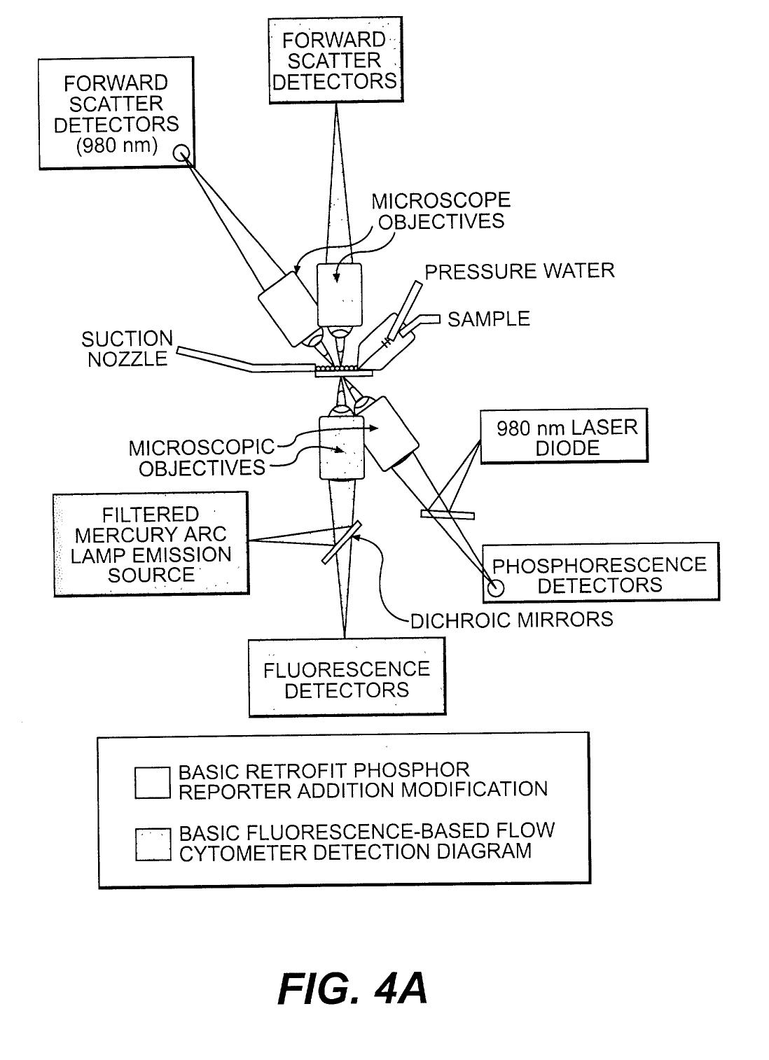

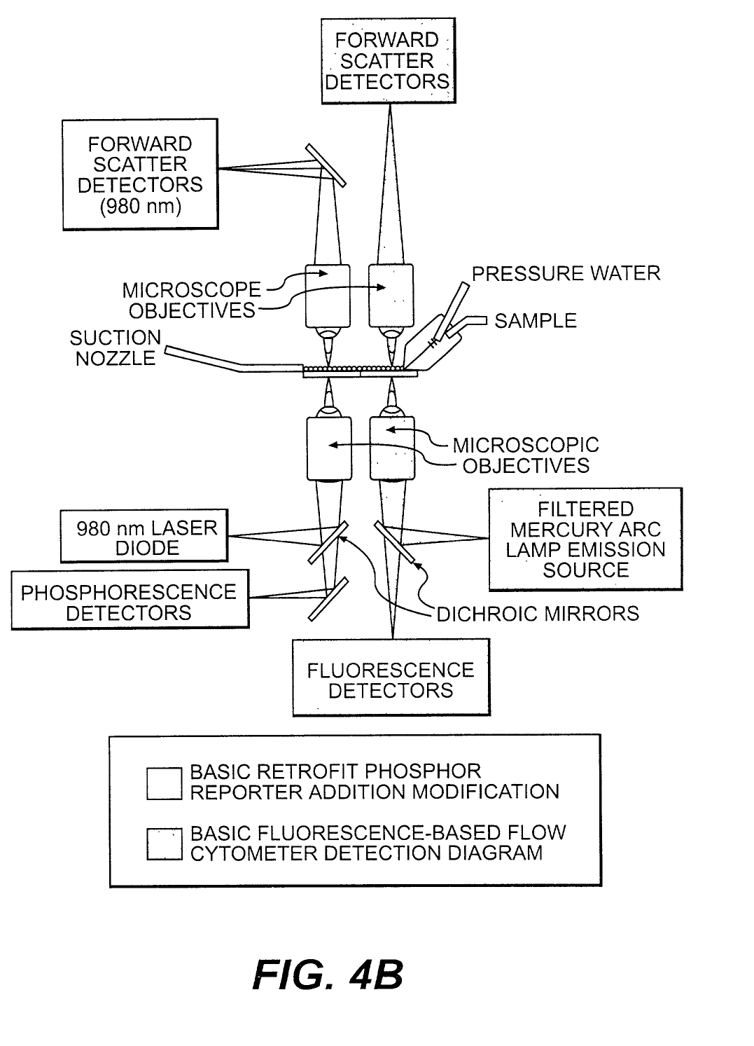

FIGS. 4A and 4B show a fluorescence-based flow cytometer modified for simultaneously assaying particle size (forward scatter detector) and phosphorescence (phosphorescence detector), as compared to the conventional particle detection and fluorescence detection. Such modifications permit flow cytometry and are compatible with combinatorial or high throughput methods.

Flow cytometer alarm algorithms can be based on the detection of a defined number of particles that exhibit: (1) a particular size; (2) a particular upconverting phosphorescence spectral signature; (3) a particular UV fluorescence spectral signature. Alternatively, these algorithms could examine the cumulative phosphorescence intensity (or rate of increase) for a given particle size and fluorescent label at a given up-conversion color.

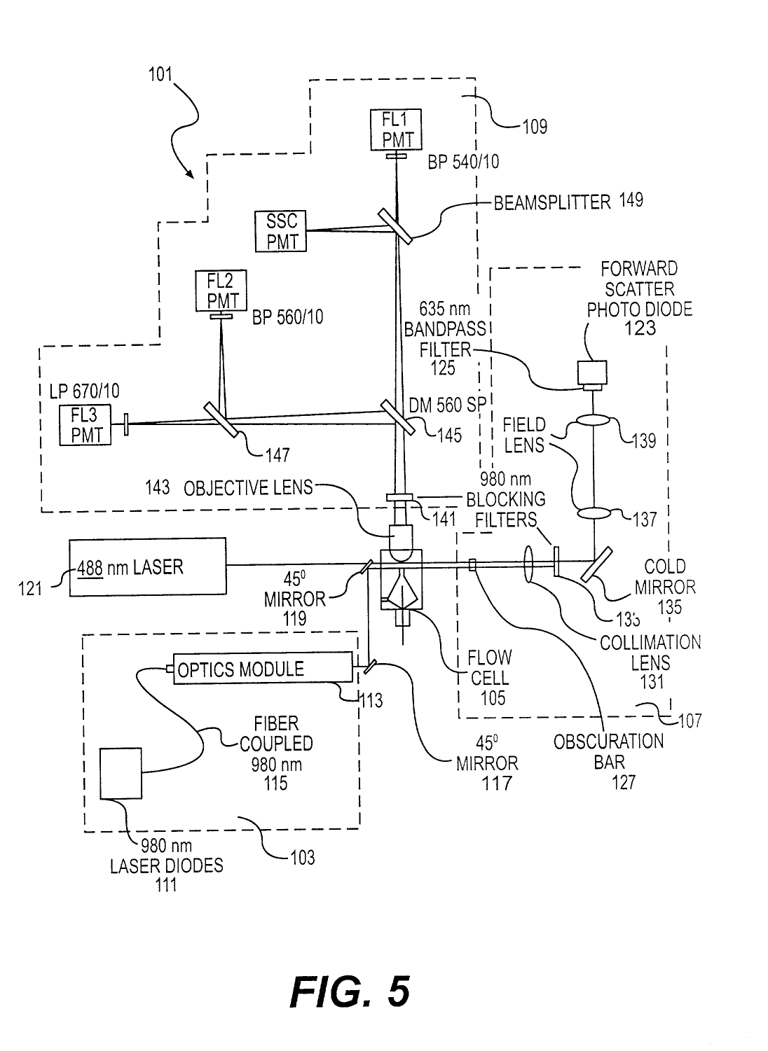

An embodiment of a flow cytometer is shown in FIG. 5. As shown, the flow cytometer 101 utilizes the flow cell and optical platform of a commercially available flow cytometer. While this embodiment may be based on other flow cytometers, a preferable flow cytometer is a Becton Dickinson FACSCalibur Flow Cytometer. Of course, the flow cell and optical platform need not be selected from a commercially available flow cytometer. In addition, the power supply of the commercially available flow cytometer may be maintained if it provides sufficient power for the flow cytometer 101. However, a commercially available flow cytometer platform provides cost effective, readily available subsystems. In addition, as described in detail below, the flow cytometer 101 comprises optics, a laser excitation source, photon detectors, and electronics to conform with the use of up-converting phosphorescent labels.

As shown in FIG. 5, a preferred embodiment comprises a laser excitation source system 103, a flow cell 105, a forward scatter detection system 107, and a side scatter detection system 109. Here, the laser excitation source system 103 may include 980 nm 1.5 W laser diodes 111 coupled to an optics module 113 for optical re-imaging by optical fibers 115. Exemplary optical fibers 115 are available from Opto Power Co. of Tucson, Ariz. having 150 μm fiber core diameters. The laser light is then directed to the flow cell 105 using two 45° mirrors 117, 119. Here, the two mirrors 117, 119 are used to direct the laser light to the flow cell 105 without removing the 488 nm laser 121 of the Becton Dickinson FACSCalibur Flow Cytometer. This allows the flow cytometer to operate using both up-converting phosphorescent labels and conventional phosphorescent labels. Here, the mirror 119 may alternatively be cold mirror. However, only one laser source is enabled during a particular measurement, because the other laser source is generally disabled. That is, the enabled laser source is determined according to the type of phosphor used.

Forward scattered light is detected by the forward scatter detection system 107 having a detection module. In the illustrated embodiment, the detector module 123 includes a detector such as a photodiode. The photodiode may be configured with a CCD camera mounted for easy visual alignment. In addition, the detector module may have a bandpass filter 125 such as a 635 nm bandpass filter. As shown in FIG. 5, forward scattered light may be directed to the detection module 123 via an obscuration bar 127, collimation lens 131, a blocking filter 133, a cold mirror 135, and field lenses 137, 139. Here, the blocking filter 133 is used to block out non-shifted light from the laser excitation source system 103. Thus, the blocking filter 133 blocks 980 nm light if the laser excitation source system 103 provides 980 nm light.

Side scattered up-shifted light is detected using the side scatter detection system 109. The side scatter detection system 109 includes fluorescent photomultiplier tubes FL 1, FL 2, FL 3 and a side scatter photomultiplier tube SSC. Here, the fluorescent photomultiplier tubes, especially FL 1, are low dark noise PMTs such as a Hamamatsu R-3896 or R-4332. Also, the side scatter photomultiplier tube SSC is infrared sensitive. In addition, each fluorescent photomultiplier tube FL 1, FL 2, FL 3 may be matched with a band pass filter corresponding to an emission wavelength of a particular phosphorescent phosphor. As illustrated in FIG. 5, 540/10 nm bandpass filter, a 560/10 nm bandpass filter, and a 670 nm long-pass filter are respectively matched with florescent photomultiplier tubes FL 1, FL 2, and FL 3.

Side scattered light may be directed to the fluorescent photomultiplier tubes using an objective lens 143, a dichroic mirror 145, and beamsplitters 147, 149. In a manner similar to the forward scatter detection system 107, a blocking filter 191 is used to block out non-shifted light from the laser excitation source system 103. Further, 980 nm notch filters (not shown) may be added to the fluorescent photomultipliers FL 1, FL 2, FL 3 for additional blocking of the 980 nm light. To direct light to the side scatter photomultiplier tube SSC, the beamsplitter 149 set at brewster's angle may be replaced by two adjustable mirrors to effectively couple the 980 nm beam into the side scatter photomultiplier tube SSC.

The signals electronic data processing is applied to the outputs of the photomultiplier tubes FL 1, FL 2, FL 3 and the detection module 123 similar to that of the Becton Dickinson FACSCalibur. However, due to the techniques of the present invention some modifications are implemented. For example, the overall amplifier gain is preferably increased, for example, by factor of 10×. The analog-to-digital converter (ADC) preferably accommodates long duration signals (i.e., those with a pulse width of up to at least 1 millisecond) and rejects short duration signals (i.e., those with a pulse width less than 3-24 microseconds). Of course, these values are exemplary and may be operator adjustable values. Thus, the baseline restorer circuit should accommodate longer duration pulses from the phosphors as compared with conventional flow cytometers. Also, the method in which the signal peak is captured and measured is modified to begin with the peak trailing edge. In accordance with specific applications, an external output cable may be desired to allow separate signal capture to digital conversion for independent analysis.

Generally, phosphor colors in the visible spectrum are preferred. Since the bandwidth of the visible spectrum is approximately 400 nm and emission bandwidths of preferred up-converting phosphors are approximately 20 nm, approximately twenty distinct colors could be detected in the visible spectrum. Here, up-converting phosphors having bandwidth other than 20 nm may be used. In addition, phosphors that up-convert to wavelengths other than visible may be used. As a result, if up-converting phosphors having emission bandwidths less than 20 nm may be used. Thus, multiplexing between larger numbers of phosphor colors may be achieved.

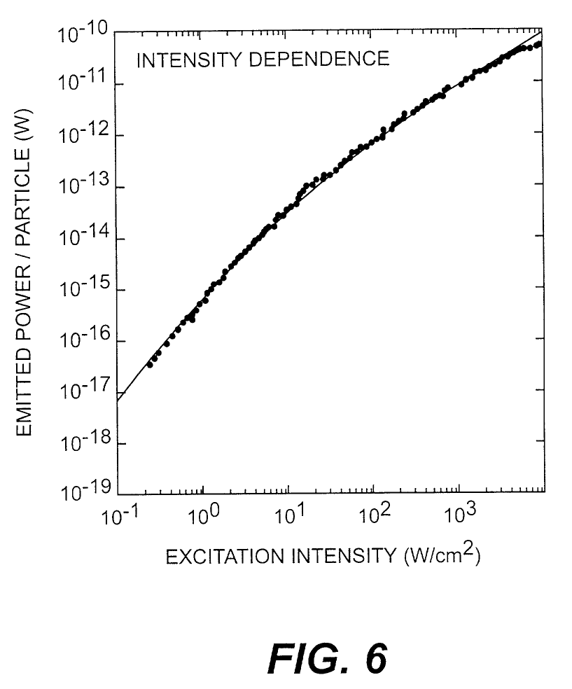

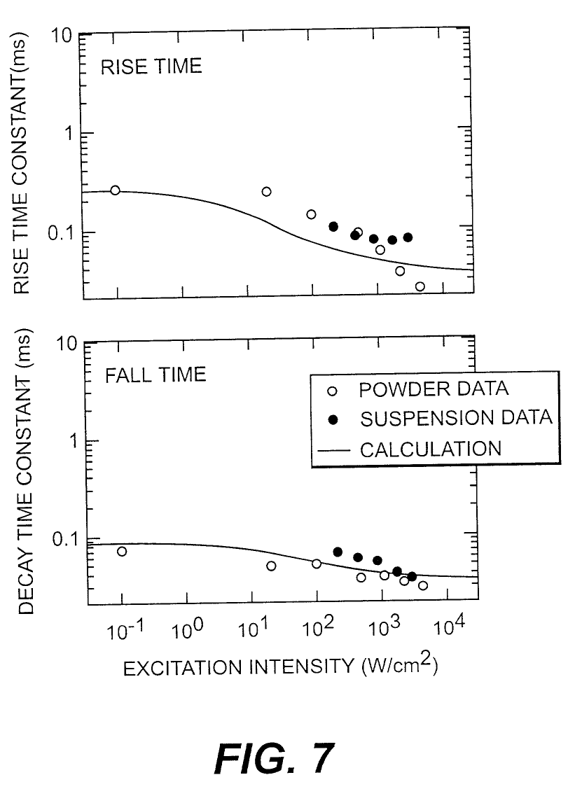

An issue regarding the performance of up-converting phosphors in flow cytometry is the time dynamics of the phosphors. Because phosphor labeled particles are in the system field of view for a short interval of time (given by the linear extent of the field divided by the flow velocity), the emission rise and fall time is a critical parameter for system performance. As shown in FIGS. 6 and 7, experimental data and theoretical modeling work has shown that (1) the phosphor time dynamics are intensity dependent, and (2) the phosphor rise and fall times are dependent on the crystal host material. Of the tested materials, oxysulfide phosphors exhibit the shortest rise and fall times. A computer model has been used in conjunction with the a theoretical model for the phosphor emission characteristics to optimize the cytometer optical system, laser spot size, and flow rate for detection of oxysulfide up-converting phosphors. The results indicate a laser focal spot size of less than 100 microns, an excitation intensity in excess of 1000 W/cm², the laser focal spot located about a spot radius upstream of the cytometer optical system, and a flow rate of about 1 m/s allows detection of single up-converting phosphor particles with a signal-to-noise ratio of about 5:1.

Another issue relevant to the overall performance of an up-converting phosphor flow cytometer is the total interrogated sample volume. To maximize light collection from each phosphor particle, the system field of view can be enlarged to collect light from a phosphor particle located anywhere along a 1 mm segment of the flow stream. Although this approach allows for single particle detection sensitivity using continuous-wave illumination sources, it has the drawback of rendering the system susceptible to background non-bound phosphor particles that may be in the same volume element. An approach to reducing this problem is to utilize a pulsed excitation source and a separate system for triggering on the presence of a capture bead in the viewed volume. This separate system can be either a second continuous-wave laser source, a Coulter principle device, or other suitable electronic system for determining the presence of a capture bead. The phosphor excitation laser can be triggered to illuminate a small volume (containing the captured particle) of the flow stream. A typical pulse duration would be several tens of microseconds, and the flow stream segment illuminated would be only several tens of microns in extent. The advantage here is that only phosphor particles in the illuminated volume would be detected, and this can be a much smaller volume than the viewed (or light collection) volume of the instrument. This latter volume being the relevant one when using continuous-wave laser illumination sources, as opposed to pulsed lasers.

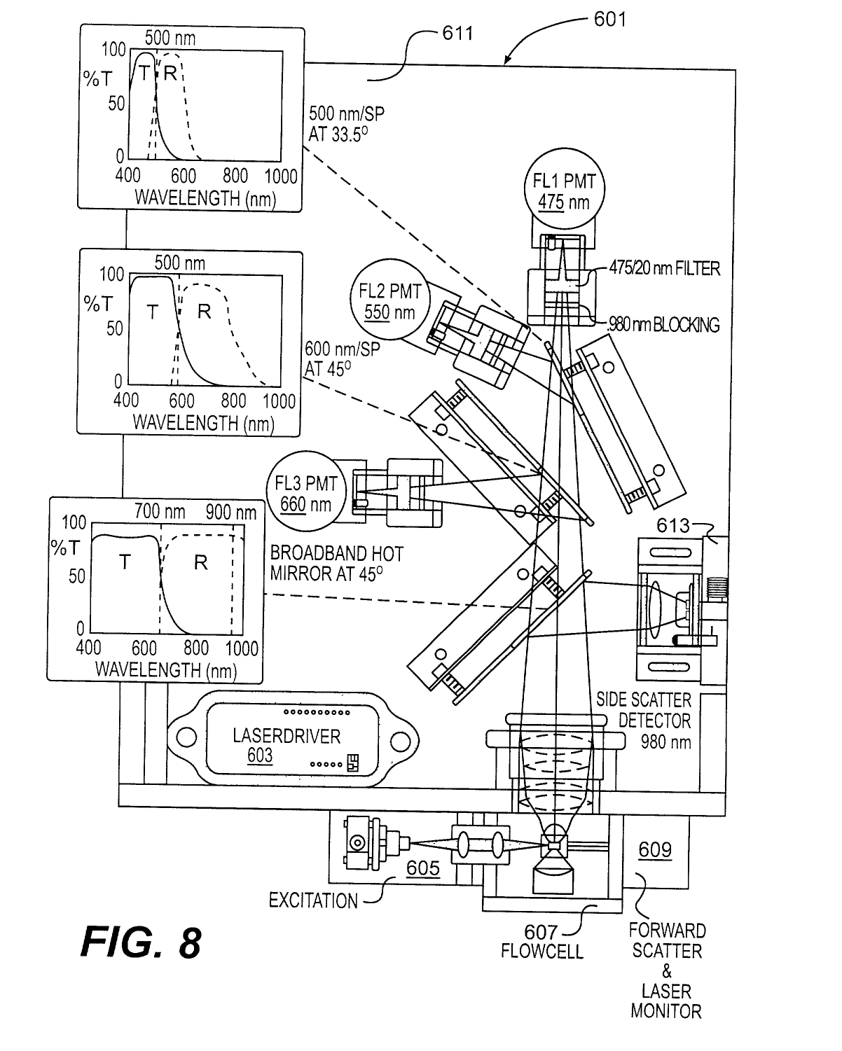

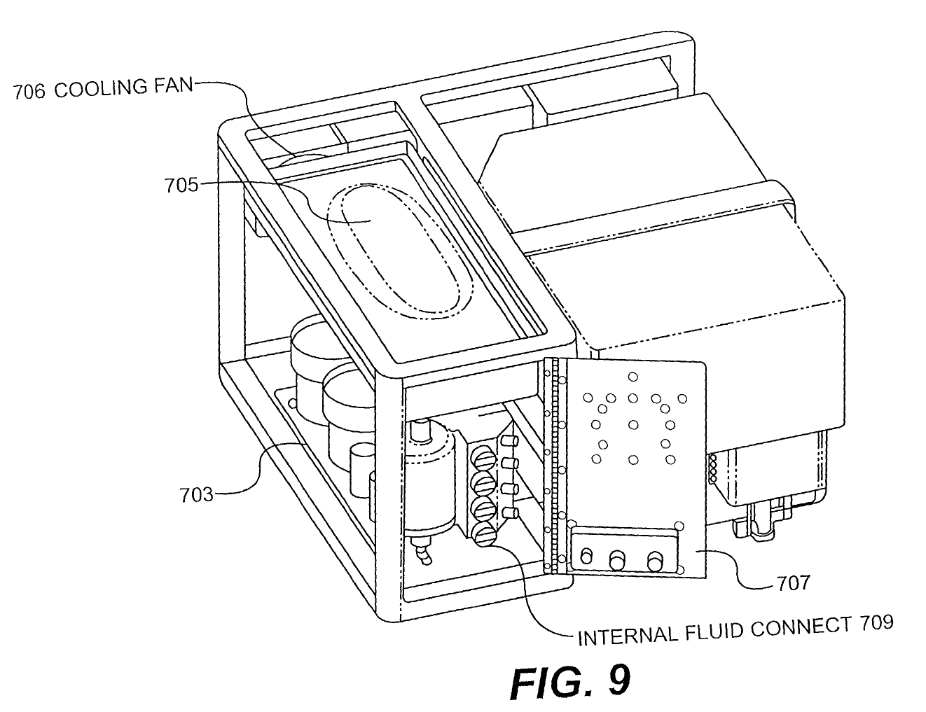

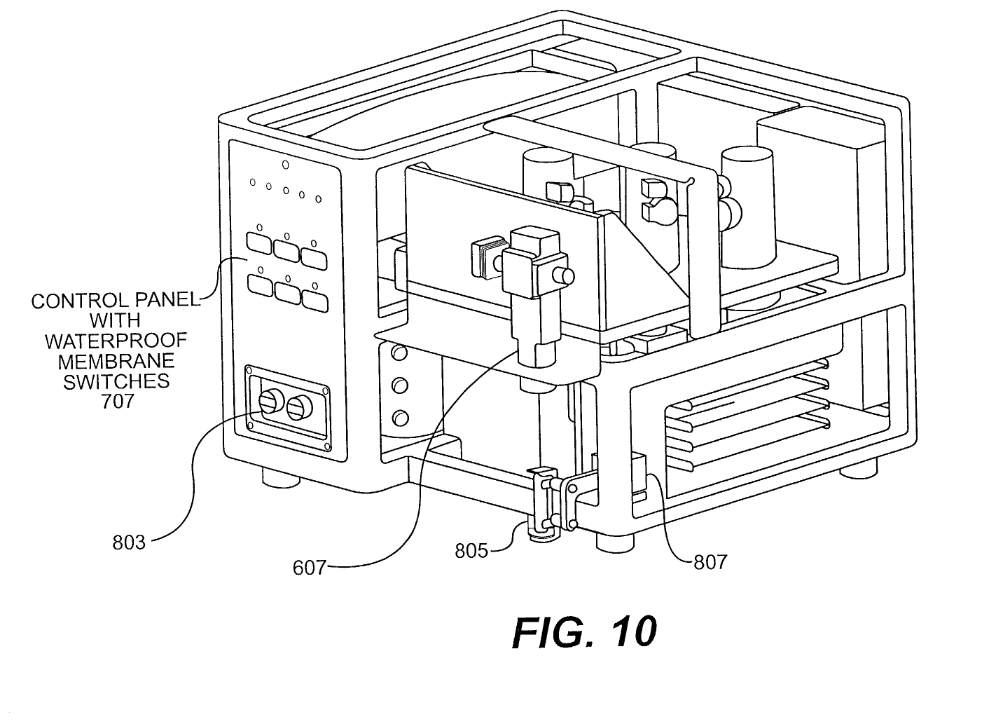

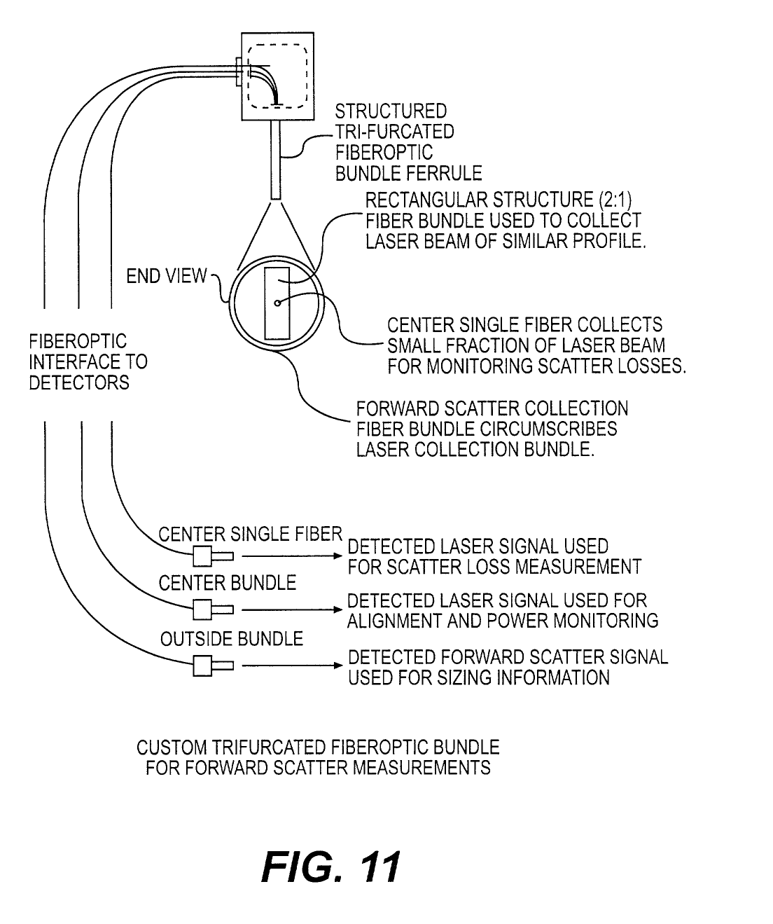

A second embodiment of the flow cytometer will now be described with reference to FIGS. 8 through FIG. 11. FIG. 8 shows a schematic of the optical plate of a flow cytometer 601. FIGS. 9 and 10 show exemplary perspective views of the flow cytometer 601. As shown in FIG. 8, the flow cytometer 601 comprises a laser driver 603, an excitation source 605, a flow cell 607, a forward scatter & laser monitor 609, and optical system 611. FIG. 11 is a schematic diagram illustrating the forward scatter of the flow cytometer of FIG. 8.

In the illustrated embodiment, the excitation source 605 is driven by the laser driver 603. Here, a 980 nm diode laser with an elliptical beam shape is used for the excitation source 605 in the flow cytometer 601. The laser diode is preferably mounted on a specially designed bracket for mechanical stability and for serving as a heat sink.

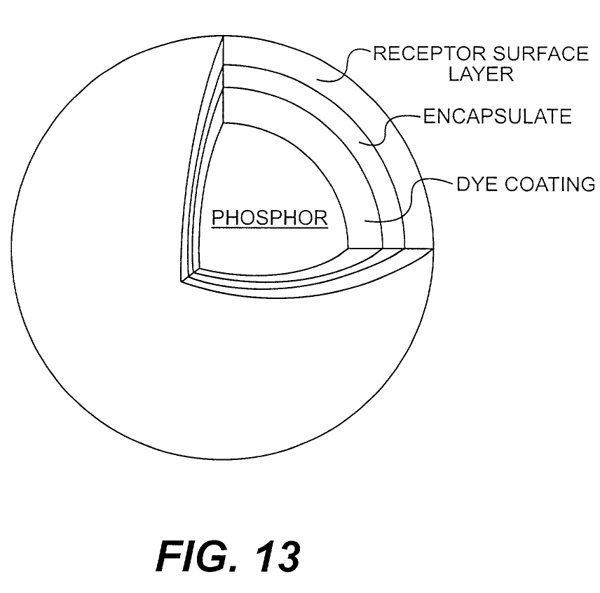

The forward scatter & laser monitor 609 has a unique concentric ringed fiber optic assembly as shown in FIG. 13. The center core of the assembly serves as an obscuration block to the incident laser beam while at the same time collecting light for the laser light power monitor (provided by a photodiode). The outer rings collect the scattered light from particles passing through the viewing orifice in the flow cell, conducting the light to a separate photodiode for sizing information.

In the optical system 611, side scatter sizing channel 613 uses a photodiode as its detector and is used for additional sizing information. Color detection is accomplished with three photomultiplier tubes with separate bandpass filters optimized for three different phosphor compositions. All of the optical components may be mounted on the optical plate using vibration isolation mounts bolted to the instrument frame.

The basic fluidics module 703 operates generally similar to that of the first embodiment. For example, the same valves and pumps are suitable. However, the sample flow rate may be reduced 6 m/s to 2 m/s to accommodate the longer phosphor excitation times according to the desired application. In addition, a bulk sheath and waste system is preferably incorporated using 20 liter reservoirs 705 for sustained operation. A cooling fan 706 preferably is provided in the fluidics module 703 to cool the electrical components. Also, all sheath fluid passes through a 0.4 micron filter to remove stray particulate matter that can cause erroneous scatter signals. Further, access is provided via control panel 707, and connections are available through connects 709, 803.

In addition, the electronics may be designed to use surface mount technology, thereby further reducing size and weight while being optimized for use with upconverting phosphors. An embedded controller may run the basic instrument functions in an exemplary system to detect fault conditions while a PC laptop computer analyzes the data and display the results. Other efficiency features may be implemented such as a sample agitator 805 and a motorized sample tube lifter 807. The sample tube lifter 807 preferably lifts the sample test tube into the sample injection port of the instrument and remove it automatically after a set timed acquisition period.

The data obtained from the samples run may be sent to a remote site, printer and stored on the hard drive or floppy disk for archival. Visual and audible alarms are provided to indicate a target detection above a preset threshold for the operator. The front control panel 707 is constructed with a membrane panel that incorporates LED indicator lights and function switches which are sealed to the outside environment for easy cleaning and decontamination. The instrument operates on 24-32 VDC or 110-240 VAC depending on the source and draws a maximum of 400 watts.

In a preferred configuration, the frame is constructed of modular aluminum extrusions bolted together, thereby allowing easy changes to the instrument, and the outside housing is fabricated from painted aluminum panels.

A number of additional features may be incorporated according to desired specification. Exemplary features include: a fully embedded computer for data acquisition and analysis, an on-board sheath fluid supply and waste containers to eliminate the 20 liter external supply and waste containers, an automated sample handling unit capable of holding and presenting 40-80 prepared samples, an array type detector to replace the bulky PMT detectors and allow for additional multiplexing capability, and a specialized flow cell to reduce fluidic consumption and increase stability for vibrations and shock.

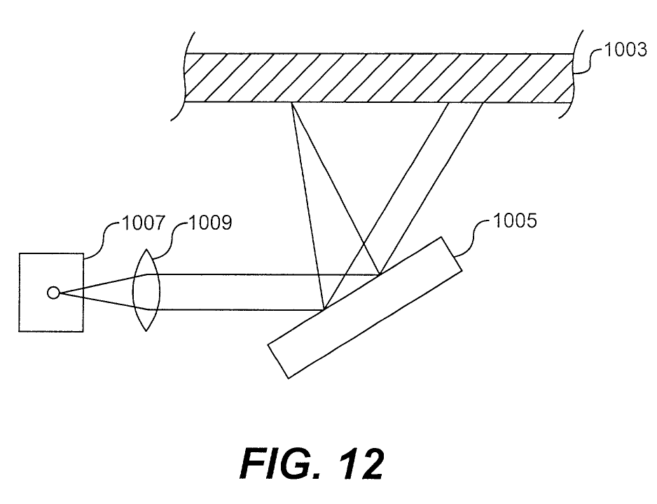

Although a three channel phosphorescent detection system has been described in the foregoing discussion, greater multiplexing may be accommodated by using a greater number of channels. In an embodiment, a configuration with a greater number of channels utilizes an array detector. FIG. 12 shows a schematic diagram of a flow cytometer configured using an array detector 1003 in combination with a dispersive optical element 1005. Here, the upshift light from the flow cell 1007 is columnated by a lens 1009. The columnated light is then directed to the dispersive optical element 1005. Here, the dispersive optical element 1005 is shown as a reflective diffraction grating. Of course, other types of dispersive optical elements may be selected, such as transmission diffraction gratings, prisms, or other suitable elements. Light from the dispersive optical element 1005 is then separated into the component wavelengths and directed to the array detector 1003. The array detector 1003 may be of any type of detector suitable to detect the incident component wavelength. However, a linear array detector is generally preferred. An exemplary linear array detector would include a 16-channel linear-array photomultiplier tube system.

Other configurations may also be used to discriminate the various phosphor colors. For example, phosphor color discrimination may be achieved with a detector and a filter wheel. In such a system, the filter wheel may be rotated to sequentially allow light emitted from a specific phosphor reports to pass. In this manner, each phosphor color may be measured serially by the detector.

Moreover, the use of up-converting phosphors in flow cytometry offers a number of advantages, thereby enabling improved performance of flow cytometers. Such advantages include higher sensitivity and lower false alarm rates. Because no background materials or sample media up-convert near infrared light, greater multiplexing potential due to the narrow line widths and larger number of spectrally unique phosphor compositions is available as compared to fluorescent reporters. Additionally, more compact instrumentation is possible due to the use of very small, electrically efficient, long lifetime diode laser excitation sources, and the up-converting phosphors are very robust for field sampling applications because they do not photobleach or chemically degrade. In addition, a 980 nm diode laser excitation source is typically much smaller and longer lasting than the 488 nm Argon laser conventionally used. This advantage may be realized because up-converting phosphors excite at 980 nm and emit at a variety of visible wavelengths in a narrow 20 nm band. Also, up-converting phosphors do not photobleach under intense irradiation. Further, any autofluorescence produced by the phosphor bead or sample when irradiated with 980 nm light will not lie within the visible up-conversion detection bands, thereby effectively eliminating background light that can limit detection sensitivity.

In addition, up-converting phosphor technology provides significant multiplexing capabilities. There are more than twenty unique phosphor colors possible, including nine spectrally unique phosphors that have been synthesized to date.

Additional Instrumental Embodiments

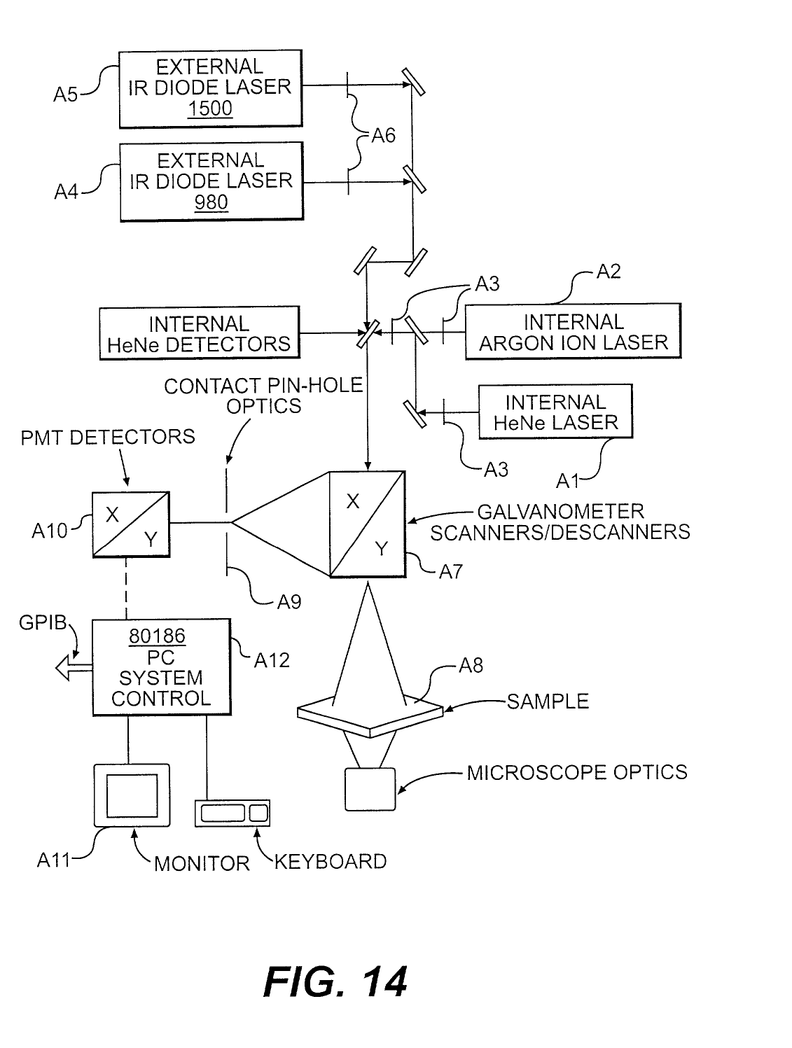

FIG. 14 is a block diagram of one embodiment of apparatus for carrying out the present invention on a sample using a microscope. In this embodiment, a standard microscope is modified to accept infrared scanning optics and image processing electronics. A suitable microscope for modification is the Zeiss model CLSM-10. However, other microscopes known in the art may also be modified.

The microscope is fitted with a HeNe laser A1 for visible imaging and an argon laser A2 for both visible and UV imaging. Both lasers are mounted internally and are individually selectable through a series of motorized shutters A3. The upconverting phosphors are excited with an externally mounted IR laser diode. In the preferred embodiment, two IR laser diodes A4 and A5, operating at two different IR wavelengths, are coupled to the microscope thereby allowing multiple phosphor labels to be identified. Laser diodes A4 and A5 are individually selectable using motorized shutters A6. When an IR beam is selected, it is routed through the microscope's galvanometrically controlled scanning mirrors A7 which scan the beam in a raster fashion. The beam passes through an objective lens (not shown) onto a sample A8 and is reflected back through the objective lens to a set of galvanometrically controlled receiving mirrors A7. Receiving mirrors A7 reflect the light onto pinhole optics A9. If the confocal mode is selected, pinhole A9 limits the detected image to the light collected from the focal plane. The light is imaged on a photomultiplier tube (PMT) Al. The thickness of the focal plane is proportional to the size of the pinhole. The scanning speed is chosen such that sufficient signal intensity is received at the PMT A10. In a preferred embodiment of this apparatus, a 20-micrometer diameter pinhole is used which results in a depth of field of about 1 micrometer. If the confocal mode is not selected, the beam is deflected around pinhole optics A9 directly to PMT A10.

Once the optical signal is converted into an electronic one, a standard, composite video signal can be developed and displayed as an image on a television monitor A11. The image may be manipulated and enhanced through standard image processing software. In a preferred embodiment of this apparatus, the software runs on an IBM 486 PC A12. The software may be used to perform averaging, filtering, edge detection and overlaying the images received from each of the different light sources.

In the confocal mode, it is possible to reconstruct a 3-dimensional view of sample A3. The reconstruction is formed by stepping through sample A8 at small intervals and making an image of the sample at each interval. The multiple sequential images are transferred to an external graphics machine (not shown) for reconstruction of the sample in 3 dimensions. These 3-D images may then be rotated to give different perspectives of the data sets, leading to a better understanding of the samples.

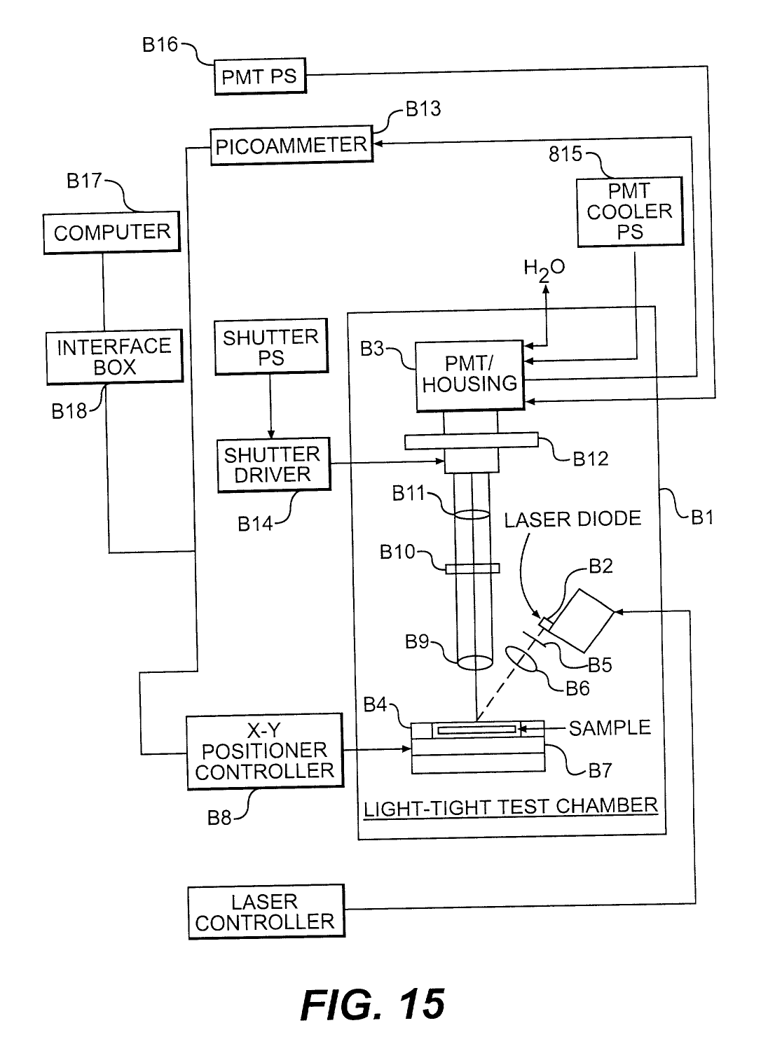

FIG. 15 is a block diagram of a reader for use in characterizing the resulting polymer beads of the present invention. Within a light-tight test chamber B1 is a near IR laser excitation source B2, a photomultiplier tube (PMT) detector B3, and a sample assay plate B4. In a preferred embodiment of this apparatus, assay plate B4 is a Terasaki HLA plate. An advantage of this plate is its small tapered sample wells, which tend to concentrate the sample material into a relatively small target area. The target area in this configuration is still larger than the diameter of the laser beam. Furthermore, it is possible that the distribution of the assay material across the bottom of the well is not even. Because of these two factors, simply aiming the laser at the center of the bottom well surface is unlikely to provide accurate readings. There are several approaches that may be used to circumvent this problem. The first approach is to defocus the laser beam sufficiently to allow a larger amount of the target area to be interrogated. However, depending upon the output of laser B2, defocussing the beam may lower the sensitivity of the apparatus to an unacceptable level. Another approach is to raster scan the laser beam across the bottom of target well. A third and preferred approach is to simply automate the scanning and data collection system.

Light from laser B2 passes through a filter B5 and is focussed by lens B6 onto an individual sample well of assay plate B4. Plate B4 is mounted on a pair of translators B7 that allow positioning in the horizontal and vertical directions. In the present configuration, translators B7 allow approximately 2.5 centimeters of travel, which is sufficient to address 3 sample wells in each direction. Translators B7 are controlled by an x-y controller B8. Controller B8 allows for either manual or computerized control.

A sample well on plate B4, when containing upconverting phosphors, will emit visible light which is collected by a lens B9, passed through a filter B10, and focussed through a lens B11 and a shutter B12 onto PMT B3. PMT B3 outputs a current, which is measured by a picoameter B13. The PMT signal is proportional to the phosphor emission intensity. Shutter B12, controlled by a shutter driver B14, provides exposure protection to PMT B3, thereby preventing damage, which may result from exposure to very intense light sources. Furthermore, overexposure of PMT B3 to light causes high dark currents, which require several hours to decrease. PMT B3 is cooled for lower dark current and noise. Associated with the PMT cooler is a water-cooled power supply B15. A power supply B16 supplies high voltage to PMT B3.

When the apparatus is operated in a computerized mode, a computer B17 regulates controller B8 through an interface box B18. Picoammeter B13 may also be connected to computer B17, thereby allowing automated data acquisition to be performed. The data acquisition procedure moves translator B7 in the x direction to a first position at which location a specified number of current readings are taken and the average is calculated. Translator B7 then moves sample B4 a predetermined distance in the x direction to a new location where new data is collected. During this process, the data is plotted in order to provide the user with an immediate visual evaluation. After the scan is completed, the data can be saved or further data processing can be performed.

Although the present invention has been described in some detail by way of illustration for purposes of clarity of understanding, it will be apparent to those of ordinary skill in the art that various modifications and equivalents can be made without departing from the spirit and scope of the invention. It should be understood that the foregoing discussion and examples merely present a detailed description of certain preferred embodiments. All patents, journal articles and other documents discussed or cited above are herein incorporated by reference in their entirety.

The claimed invention is:

1. A method for polymerization, comprising the steps of:
combining a monomer with at least one labeled catalyst particle, wherein said labeled catalyst particle comprises at least one up-converting phosphor label and a catalyst,
wherein said up-converting phosphor label identifies said catalyst, and
comprises at least one rare earth ion and at least one phosphor host material;
and
polymerizing said monomer to form a polymer bead surrounding said labeled catalyst particle.

2. The method of claim 1, wherein said monomer is combined with at least two labeled catalyst particles.

3. The method of claim 2, wherein the wavelength of the excitation radiation of a first phosphor is about equal to the wavelength of the excitation radiation of a second phosphor, and wherein the wavelength of the emission radiation of said first phosphor is different from the wavelength of the emission radiation of said second phosphor.

4. The method of claim 2, wherein the wavelength of the excitation radiation of a first phosphor is different from the wavelength of the excitation radiation of a second phosphor, and wherein the wavelength of the emission radiation of said first phosphor is about the same as the wavelength of the emission radiation of said second phosphor.

5. The method of claim 2, wherein the wavelength of the excitation radiation of a first phosphor is different from the wavelength of the excitation radiation of a second phosphor, and wherein the wavelength of the emission radiation of the first phosphor is different from the wavelength of the emission radiation of the second phosphor.

6. The method of claim 1, further comprising, after the polymerizing step, a step of sorting said polymer beads.

7. The method of claim 6, wherein the sorting step comprises:
illuminating at least one polymer bead with excitation radiation;
detecting emission radiation of at least one emission wavelength; and
separating said polymer beads.

8. The method of claim 7, wherein said excitation radiation is near infrared radiation and said emission radiation is visible radiation.

9. The method of claim 7, wherein said up-converting phosphor label comprises a rare earth ion selected from the group consisting of ytterbium and erbium.

10. The method according to claim 9, wherein said up-converting phosphor label comprises sodium yttrium fluoride ytterbium erbium or yttrium ytterbium erbium oxysulfide.

11. The method of claim 1, wherein said phosphor host material is selected from the group consisting of barium-yttrium-fluoride, sodium yttrium fluoride (NaYF₄), lanthanum fluoride (LaF₃), lanthanum oxysulfide, yttrium oxysulfide, yttrium fluoride (YF₃), yttrium gallate, yttrium aluminum garnet, gadolinium fluoride (GdF₃), barium yttrium fluoride (BaYF₅, BaY₂F₈), gadolinium oxysulfide, lanthanum oxide, gadolinium oxide, alumina, aluminum oxysulfide, aluminum trifluoride, magnesium difluoride, magnesium dichloride and yttrium oxide; and
wherein said rare earth ion is selected from the group consisting of an erbium ion, neodymium ion, a thulium ion, a holmium ion and a praseodymium ion.

12. The method of claim 1, wherein said monomer is polymerized in a gas phase or slurry phase, or via emulsion polymerization.

13. The method of claim 1, wherein said monomer is selected from the group consisting of:
ethylene, propylene, cis-2-butene, butadiene, 1-hexene, 1-octene, 1-butene, 3 methyl-1-butene, 1,3-butadiene, 1-pentene, 4-methyl-1-pentene, 1-hexene, 4-methyl-1-hexene, 1,4-hexadiene, 1,5-hexadiene, 1-octene, 1,6-octadiene, 1-nonene, 1-decene, 1,4-dodecadiene, 1-hexadecene, 1-octadecene, cyclopentene, 3-vinylcyclohexene, 5-vinyl-2-norbomene, 5-ethylidene-2-norbomene, dicyclopentadiene, 4-vinylbenzocyclobutane, tetracyclododecene, dimethano-octahydronaphthalene, 7-octenyl-9-borabicyclo-(3,3,1)nonane, styrene, o-methylstyrene, m-methylstyrene, p-methylstyrene, p-tert-butylstyrene, m-chlorostyrene, p-chlorostyrene, p-fluorostyrene, indene, 4-vinylbiphenyl, acenaphthalene, vinylfluorene, vinylanthracene, vinylphenanthrene, vinylpyrene, methylmethacrylate, ethylacrylate, vinyl silane, phenyl silane, trimethylallyl silane, acrylonitrile, maleimide, vinyl chloride, vinylidene chloride, tetrafluoroethylene, isoprene, isobutylene, carbon monoxide, acrylic acid, 2-ethylhexylacrylate, methyl acrylate, methyl methacrylate, methacrylonitrile, methacrylic acid, vinyl acetate, norbornene, norbornadiene, and mixtures thereof.

14. The method of claim 1, wherein said catalyst is selected from the group consisting of Zieglar-Natta catalysts, metallocene catalysts, stereospecific catalysts, constrained geometry catalysts, single-site catalysts, late transition metal single-site catalysts, free radial initiators, living free radical initiators, cationic initiators, anionic initiators, and mixtures thereof.

15. The method of claim 1, wherein said labelled catalyst particle further comprises a solid support material selected from the group consisting of a porous resinous material and a solid inorganic oxide.

16. A method for screening a library of catalysts, comprising the steps of:
combining a monomer with at least two labeled catalyst particles, wherein each labeled catalyst particle comprises an up-converting phosphor label and a catalyst,
wherein said up-converting phosphor label identifies a particular catalyst, and comprises at least one rare earth ion and at least one phosphor host material;
polymerizing said monomer to form a polymer bead surrounding said labeled catalyst particle; and
sorting at least two of said polymer beads.

17. The method of claim 16, wherein the sorting step is accomplished through the use of a cytometer.

18. The method of claim 16, wherein the sorting step comprises distinguishing said polymer beads based upon size.

19. The method of claim 16, wherein the sorting step comprises:
illuminating at least one polymer bead with excitation radiation;
detecting emission radiation of at least one emission wavelength; and
classifying the polymer beads based on the up-converting phosphor label.

20. A method for screening a solid support material for a supported catalyst, comprising the steps of:
combining a monomer with at least one labeled catalyst particle, wherein said labeled catalyst particle comprises an up-converting phosphor label and a catalyst,
wherein said up-converting phosphor label identifies a particular support material, and comprises at least one rare earth ion and at least one phosphor host material; and
polymerizing said monomer to form a polymer bead surrounding said labeled catalyst particle.

21. A composition comprising a labeled catalyst particle which comprises an up-converting phosphor label and a catalyst, wherein said up-converting phosphor label comprises at least one rare earth ion and at least one phosphor host material.

22. The composition of claim 21, further comprising a solid support material selected from the group consisting of a porous resinous material and a solid inorganic oxide.

23. The composition of claim 21, wherein said up-converting phosphor further comprises at least one compound selected from the group consisting of:
Na(YₓYb_{y}Er_{z})F₄: wherein x is 0.7 to 0.9, y is 0.09 to 0.29, and z is 0.05 to 0.01;
Na(YₓYb_{y}Ho_{z})F₄: wherein x is 0.7 to 0.9, y is 0.0995 to 0.2995, and z is 0.0005 to 0.001;
Na(YₓYb_{y}Pr_{z})F₄: wherein x is 0.7 to 0.9, y is 0.0995 to 0.2995, and z is 0.0005 to 0.001;
Na(YₓYb_{y}Tm_{z})F₄: wherein x is 0.7 to 0.9, y is 0.0995 to 0.2995, and z is 0.0005 to 0.001; and
(YₓYb_{y}Er_{z})O₂S: wherein x is 0.7 to 0.9, y is 0.05 to 0.12; z is 0.05 to 0.12.

24. A composition of claim 21, wherein said up-converting phosphor further comprises at least one compound selected from the group consisting of:
(Y_{0.80}Yb_{0.18}Er_{0.02})F₃; (Y_{0.87}Yb_{0.13}Tm_{0.001})F; (Y_{0.80}Yb_{0.198}Ho_{0.002})F_{3;}
(Gd_{0.08}Yb_{0.18}Er_{0.02})F₃; (Gd_{0.87}Yb_{0.13}Tm_{0.001})F₃; (Gd_{0.80}Yb_{0.198}Ho_{0.002})F_{3;}
(Y_{0.86}Yb_{0.08}Er_{0.6})₂O₂S; (Y_{0.87}Yb_{0.13}Tm_{0.001})₂O₂S; (Y_{0.08}Yb_{0.198}Ho_{0.0022})O₂S;
(Gd_{0.86}Yb_{0.08}Er_{0.06})₂O₂S; (Gd_{0.87}Yb_{0.13}Tm_{0.001})₂O₂S; and
(Gd_{0.08}Yb_{0.198}Ho_{0.002})₂O₂S.

25. The composition of claim 21, wherein said catalyst is selected from the group consisting of Zieglar-Natta catalysts, metallocene catalysts, stereospecific catalysts, constrained geometry catalysts, single-site catalysts, late transition metal single-site catalysts, free radical initiators, living free radical initiators, cationic initiators, anionic initiators, and mixtures thereof.
